(19) United States
(12) Patent Application Publication
(10) Pub. No.: US 2018/0153990 A1
(43) Pub. Date: Jun. 7, 2018

(54) PCA1 PROTEIN AND METHODS OF TREATING PNEUMOCYSTIS PNEUMONIA INFECTION

(71) Applicant: University of Rochester, Rochester, NY (US)

(72) Inventors: Francis Gigliotti, Rochester, NY (US); Terry Wright, Rochester, NY (US)

(21) Appl. No.: 15/826,297

(22) Filed: Nov. 29, 2017

Related U.S. Application Data
(60) Provisional application No. 62/427,574, filed on Nov. 29, 2016.

(51) Int. Cl. A61K 39/395; C12N 1/16; C07K 16/14; A61P 11/00

(52) U.S. Cl. CPC A61K 39/39575; A61P 11/00; C07K 16/14; C12N 1/16

(57) ABSTRACT

The present invention relates to a method of treating Pneumocystis pneumonia infection in a subject. This method involves administering to a subject having a Pneumocystis pneumonia infection one or more antibodies that bind specifically to a Pneumocystis cross-reactive antigen 1 (PCA1) protein under conditions effective to treat the Pneumocystis infection in the subject. Another aspect of the present invention relates to a method of treating a subject at risk for Pneumocystis pneumonia infection. A further aspect of the present invention relates to an isolated protein or polypeptide comprising the amino acid sequence of SEQ ID NO:1. Another aspect of the present invention relates to a pharmaceutical composition comprising the isolated protein or polypeptide of the present invention and a pharmaceutically acceptable carrier.

This application claims the priority benefit of U.S. Provisional Patent Application Ser. No. 62/427,574 filed Nov. 29, 2016, which is hereby incorporated by reference in its entirety.

This invention was made with Government Support under AI023302, HL092797, AI007464, HD068373 awarded by the National Institutes of Health. The Government has certain rights in the invention.

### FIELD OF THE INVENTION

The present invention relates to PCA1 protein and methods of treating pneumocystis pneumonia infection.

### BACKGROUND OF THE INVENTION

Pneumocystis pneumonia (PcP) is a common opportunistic infection, with 10,000 estimated cases each year in the U.S. and more than 400,000 cases worldwide (Kovacs et al., “New Insights Into Transmission, Diagnosis, and Drug Treatment of Pneumocystis Carinii Pneumonia,”Jama 286:2450-60 (2001); Brown et al., “Hidden Killers: Human Fungal Infections,” Sci. Transl. Med. 4(165):165rv13 (2012)). The need for admission to the intensive care unit and ventilator support is common. Mortality rates of up to 40% are typical in high-risk patient populations despite first-line treatment. PcP affects immunosuppressed hosts, with cancer patients and organ transplant recipients accounting for the majority of cases in developed countries (Maini et al., “Increasing Pneumocystis Pneumonia, England, U K, 2000-2010,” Emerg. Infect. Dis. 19:386-92 (2013); Saltzman et al., “Clinical Conditions Associated With PCP in Children,” Pediatr. Pulmonol. 47:510-6 (2012)). Patients receiving immunomodulatory agents as well as those with pre-existing lung disease represent growing patient populations at risk of developing PcP. These factors contributed to an overall increase in the incidence of PcP in England from 2000-2010, despite a decline in the number of HIV-associated cases during that time period (Maini et al., “Increasing Pneumocystis Pneumonia, England, U K, 2000-2010,” Emerg. Infect. Dis. 19:386-92 (2013)).

While effective chemoprophylaxis for PcP exists, half of all PcP cases occur in those prescribed adequate prophylaxis, most commonly a result of noncompliance (Morris et al., “Current Epidemiology of Pneumocystis Pneumonia,” Emerg. Infect. Dis. 10:1713-20 (2004)). Another threat to the effectiveness of chemoprophylaxis is the potential for the development of resistance to trimethoprim-sulfamethoxazole, unquestionably the most effective prophylactic and therapeutic agent (Huang et al., “Dihydropteroate Synthase Gene Mutations in Pneumocystis and Sulfa Resistance,” Emerg. Infect. Dis. 10:1721-8 (2004)).

Mortality rates have changed little over the past few decades, emphasizing the need for additional treatment options. Immunization, passive or active, is a desirable approach that has not yet proved successful. Active immunization of children and adults with cancer against bacterial and viral pathogens during the initial phases of chemotherapy has been shown to protect them through periods of immunosuppression (Feldman et al., “Risk of Haemophilus Influenzae Type B Disease in Children With Cancer and Response of Immunocompromised Leukemic Children to a Conjugate Vaccine,” J. Infect. Dis. 161:926-31 (1990); Nordoy et al., “Cancer Patients Undergoing Chemotherapy Show Adequate Serological Response to Vaccinations Against Influenza Virus and Streptococcus Pneumoniae,” Med. Oncol. 19:71-8 (2002); LaRussa et al., “Varicella Vaccine for Immunocompromised Children: Results of Collaborative Studies in the United States and Canada,” J. Infect. Dis. 174(Suppl 3):S320-3 (1996)). Pneumocystis (Pc) is an attractive target for vaccine-based prevention since patient populations at risk for PcP can often be identified prior to becoming immunosuppressed.

Active immunization with whole organisms is uniformly protective in animal models of PcP (Harmsen et al., “Active Immunity to Pneumocystis Carinii Reinfection in T-Cell-Depleted Mice,” Infect. Immun. 63:2391-5 (1995); Garvy et al., “Protection Against Pneumocystis Carinii Pneumonia by Antibodies Generated From Either T Helper 1 or T Helper 2 Responses,” Infect. Immun. 65:5052-6 (1997); Pascale et al., “Intranasal Immunization Confers Protection Against Murine Pneumocystis Carinii Lung Infection,” Infect. Immun. 67:805-9 (1999)). However, the antigenic profile of Pc infecting each host is so distinct from that of Pc infecting any other host species that cross protective immunity is not induced. For example, immunization of mice with mouse-derived Pc (P. murina) protects them from subsequent infection, while immunization with ferret-derived Pc fails to protect (Gigliotti and Harmsen, “Pneumocystis Carinii Host Origin Defines the Antibody Specificity and Protective Response Induced by Immunization,” J. Infect. Dis. 176:1322-6 (1997)).

The inability to cultivate the organism is a further impediment to vaccine development. An alternative approach is to use molecular techniques to develop a subunit vaccine, especially one that contains cross-reactive epitopes. Such antigens are rare but do exist (Gigliotti and Harmsen, “Pneumocystis Carinii Host Origin Defines the Antibody Specificity and Protective Response Induced by Immunization,” J. Infect. Dis. 176:1322-6 (1997); Gigliotti et al., “Development of Murine Monoclonal Antibodies to Pneumocystis Carinii,” J. Infect. Dis. 154:315-22 (1986)). Thus far, the efficacy of subunit vaccines for Pc has not matched that observed with whole cell vaccination.

Further, with increasing utilization of immunomodulatory agents, the pool of patients at risk of developing Pcp will likely increase (Roux et al., “Update on Pulmonary Pneumocystis Jirovecii Infection in Non-HIV Patients,” Med Mal. Infect. 44:185-98 (2014)). For example, soon after the introduction of rituximab, a monoclonal antibody that targets the CD20 antigen on B lymphocytes, reports of Pcp associated with B cell depletion began to appear in the literature (Martin-Garrido et al., “Pneumocystis Pneumonia in Patients Treated with Rituximab,” Chest 144:258-65 (2013)).

Opsonization of microorganisms is important for clearance by phagocytes. Different proteins can act as opsonins. The role of opsonins in clearance of fungi has not been well studied; however, there is some experimental support for their importance. For example, the fungal pathogen Candida albicans was shown to be more efficiently phagocytosed in the presence of mannose-binding lectin (“MBL”) compared to conditions when the opsonin MBL was absent (Brouwer et al., “Mannose-Binding Lectin (MBL) Facilitates Opsonophagocytosis of Yeasts but not of Bacteria Despite MBL Binding,” J. Immunol. 180:4124-32 (2008)). Two opsonins shown to affect clearance of Pneumocystis are complement and antibody (Wells et al., “Complement and Fc Function are Required for Optimal Antibody Prophylaxis Against Pneumocystis Carinii Pneumonia,” Infect. Immun. 74:390-3 (2006); Gigliotti et al., “Passive Intranasal Monoclonal Antibody Prophylaxis Against Murine Pneumocystis Carinii Pneumonia,” Infect. Immun. 70:1069-74 (2002)).

Standardized assays to measure phagocytosis of Pneumocystis have only recently been developed. As a result, there is only limited experimental support for antibody acting in concert with macrophages to clear Pneumocystis (Wells et al., “Complement and Fc Function are Required for Optimal Antibody Prophylaxis Against Pneumocystis Carinii Pneumonia,” Infect. Immun. 74:390-3 (2006); Wang et al., “Immune Modulation With Sulfasalazine Attenuates Immunopathogenesis but Enhances Macrophage-Mediated Fungal Clearance During Pneumocystis Pneumoni,” PLoS Pathog. 6:e1001058 (2010); Gigliotti et al., “Passive Intranasal Monoclonal Antibody Prophylaxis Against Murine Pneumocystis Carinii Pneumonia,” Infect. Immun. 70:1069-74 (2002); Limper et al., “The Role of Alveolar Macrophages in Pneumocystis Carinii Degradation and Clearance From the Lung,” J. Clin. Invest. 99:2110-7 (1997)).

In addition to the specific effects of antibody, antibody has also been shown to have non-specific immune modulatory effects as exemplified by their use in diseases like idiopathic thrombocytopenic purpura or Kawasaki disease (Luzi et al., “Intravenous IgG: Biological Modulating Molecules,” J. Biol. Regul. Homeost. Agents 23:1-9 (2009); Gupta et al., “Cytokine Modulation with Immune Gamma-Globulin in Peripheral Blood of Normal Children and its Implications in Kawasaki Disease Treatment,” J. Clin. Immunol. 21:193-9. (2001); Kazatchkine et al., “Immunomodulatory Effects of Intravenous Immunoglobulins,” Ann. Med. Interne. (Paris) 151(Suppl 1):1S13-8 (2000); Wolf and Eibl, “Immunomodulatory Effect of Immunoglobulins,” Clin. Exp. Rheumatol. 14(Suppl 15):S17-25 (1996); Mazer et al., “Immune Supplementation and Immune Modulation with Intravenous Immunoglobulin,” J. Allergy Clin. Immunol. 116:941-4 (2005); Samuelsson et al., “Anti-Inflammatory Activity of IVIG Mediated Through the Inhibitory Fc Receptor,” Science 291:484-6 (2001)). This effect of antibody or immunoglobulin (Ig) could prove valuable in the management of Pcp because of the prominent inflammatory component associated with Pcp.

Macrophages are important phagocytic cells. Studies of macrophage biology have demonstrated them to be complex cells whose function varies with inducible phenotype. Classically activated macrophages (CAM) or M1 macrophages have an inflammatory phenotype in response to exposure to lipopolysaccharide (LPS) and interferon gamma. In contrast, alternatively activated macrophages (AAM) or M2 macrophages are pro-resolution and/or anti-inflammatory and can be programmed via multiple mechanisms including exposure to Interleukin 4 and 13 (IL-4/IL-13) or antigen-antibody immune complexes (Tarique et al., “Phenotypic, Functional and Plasticity Features of Classical and Alternatively Activated Human Macrophages,” Am. J. Respir. Cell Mol. Biol. 53(5):676-88 (2015); Arango et al., “Macrophage Cytokines: Involvement in Immunity and Infectious Diseases,” Front Immunol. 5: 491 (2014)).

However, the effect of specific antibody on modulation of macrophage phenotype during Pcp has not been evaluated.

The present invention is directed to overcoming deficiencies in the art.

### SUMMARY OF THE INVENTION

One aspect of the present invention relates to a method of treating Pneumocystis pneumonia infection in a subject. This method involves administering to a subject having a Pneumocystis pneumonia infection one or more antibodies that bind specifically to a Pneumocystis cross-reactive antigen 1 (PCA1) protein under conditions effective to treat the Pneumocystis infection in the subject.

Another aspect of the present invention relates to a method of treating a subject at risk for Pneumocystis pneumonia infection. This method involves administering to a subject at risk for Pneumocystis pneumonia infection a Pneumocystis protein or polypeptide comprising the amino acid sequence of SEQ ID NO:1 under conditions effective to prevent the Pneumocystis infection.

A further aspect of the present invention relates to an isolated protein or polypeptide comprising the amino acid sequence of SEQ ID NO:1.

Another aspect of the present invention relates to a pharmaceutical composition comprising the isolated protein or polypeptide of the present invention and a pharmaceutically acceptable carrier.

A protective monoclonal antibody (Mab), 4F11, that is cross reactive with other Pc species, including P. jiroveci was previously identified (Gigliotti and Harmsen, “Pneumocystis Carinii Host Origin Defines the Antibody Specificity and Protective Response Induced by Immunization,” J. Infect. Dis. 176:1322-6 (1997), which is hereby incorporated by reference in its entirety). Active immunization with a 142-amino acid polypeptide (A12) that contains a 4F11 epitope elicits a protective response, decreasing organism burden and lung inflammation (Wells et al., “Active Immunization Against Pneumocystis Carinii With a Recombinant P. Carinii Antigen,” Infect Immun 74:2446-8 (2006), which is hereby incorporated by reference in its entirety).

As described herein, the full-length cDNA from which the A12 C-terminal polypeptide was derived has now been isolated and partially characterized. Based on the findings described herein, the name Pneumocystis cross-reactive antigen 1 (“PCA1”) has been used for this molecule. Here, it is shown that active immunization with the N-terminal half of PCA1 protected against infection in a CD4⁺ T cell-depleted mouse model of PcP. Furthermore, antibody generated from the immunization of mice with this protein also recognizes epitopes on the surface of the human pathogen P. jirovecii, highlighting the possibility of developing PCA1 as a human vaccine candidate.

Further, as described above, it has been demonstrated that antibody treatment can improve clearance of Pneumocystis (Gigliotti et al., “Passive Intranasal Monoclonal Antibody Prophylaxis Against Murine Pneumocystis Carinii Pneumonia,” Infect. Immun. 70:1069-74 (2002); Gigliotti and Hughes, “Passive Immunoprophylaxis With Specific Monoclonal Antibody Confers Partial Protection Against Pneumocystis Carinii Pneumonitis in Animal Models,” J. Clin. Invest. 81:1666-8 (1988); Empey et al., “Passive Immunization of Neonatal Mice Against Pneumocystis Carinii f sp. Muris Enhances Control of Infection Without Stimulating Inflammation,” Infect. Immun. 72:6211-20 (2004), which are hereby incorporated by reference in their entirety). However, the mechanism of this effect is not fully understood and the effect of specific antibody on modulation of macrophage phenotype during Pcp has not been evaluated.

It has also been demonstrated that sulfasalazine (SSZ), an anti-inflammatory drug acts, at least in part, by inhibiting nuclear factor kappa beta (NF-κB), shifts alveolar macrophages to an AAM or M2 phenotype, and improves clearance of Pneumocystis while at the same time reducing the inflammatory lung injury associated with Pcp (Wang et al., “Immune Modulation With Sulfasalazine Attenuates Immunopathogenesis but Enhances Macrophage-Mediated Fungal Clearance During Pneumocystis Pneumonia. PLoS Pathog. 6:e1001058 (2010), which is hereby incorporated by reference in its entirety).

Described herein is the effect of passive immunization on macrophage phenotype and subsequent clearance of Pneumocystis. It was hypothesized that the efficacy of passive immunization could be enhanced by the addition of SSZ by providing an additional signal to shift alveolar macrophages to a pro-phagocytic, anti-inflammatory phenotype. It was reasoned that administration of antibody to Pneumocystis to mice with Pcp would result in the in vivo formation of immune complexes which, when taken up by macrophages, would promote a shift to an M2 phenotype, resulting in an anti-inflammatory environment which would allow for an enhanced resolution of Pcp.

### BRIEF DESCRIPTION OF THE DRAWINGS

FIGS. 1A-C are an alignment of human (hPc), mouse (mPc), and rat (rPc) Pneumocystis PCA1. The cysteine residues conserved in all three proteins are highlighted. Other amino acids that are conserved in all three sequences are also highlighted. Amino acids that are conserved in human Pneumocystis PcA1 and at least one of the other proteins are highlighted. The Genbank accession numbers for each protein are listed at the top of the page.

FIGS. 2A-2B are graphs showing that Pca1 immunization reduces organism burden in a dose-dependent trend. FIG. 2A shows that mice immunized with Pca1 fusion protein and detectable Pc by qPCR (n=37, triangles) had reduced organism burden compared to immunization with the fusion partner (n=10, circles) or an irrelevant protein (n=18, squares). Data points represent log transformation of qPCR with the limit of assay detection marked by dotted line. Data is from 6 color-coded experiments. FIG. 2B shows that increasing doses of Pca1 immunization resulted in an increased number of mice protected from infection (**p<0.01, *p<0.05 compared to negative control). Data is from 2 pooled experiments.

FIGS. 3A-3B are graphs showing Pca1-specific antibody development in immunized mice. FIG. 3A shows pooled sera from five mice bled three weeks after completing a three dose immunization series of 80 μg of PCA1 fusion protein (high dose Pca1, circles) demonstrated antigen-specific antibody as measured by ELISA to a Pca1 polypeptide. Pooled sera from five mice immunized with only 10 μg of the Pca1 fusion protein (low dose Pca1, diamonds) or 80 μg of the fusion partner only (negative control, squares) did not demonstrate significant antibody development above PBS-immunized mice (sham, triangles). FIG. 3B shows Pca1-specific antibody remained detectable in high dose Pca1-immunized mice approximately six weeks following CD4⁺ T cell depletion and subsequent exposure to Pc, corresponding to approximately nine weeks after last immunization. Data represents mean+/−SEM of five individual animals in each group.

FIGS. 4A-4D are images showing that Pca1 antisera recognizes both mouse and human Pc cysts by IFA. Antisera from mice immunized with the Pca1 fusion protein bound not only to the surface of mouse species-specific Pc (P. murina) cysts (FIG. 4A) but also rat-specific P. carinii (FIG. 4B) and the human pathogen P. jirovecii (FIG. 4C). No such binding was observed with fusion partner-immunized antisera (FIG. 4D).

FIGS. 5A-4C are graphs showing accelerated recovery from PcP in mice treated with anti-Pneumocystis antibody, SSZ, or a combination of both. Pneumocystis-infected SCID mice were immune reconstituted with wild-type splenocytes. Treatments were started (FIG. 5A, arrow) eight days post-reconstitution when the mice displayed obvious signs of PCP (>10% body weight loss and >25% increase in respiratory rate). Body weight loss (FIG. 5A) was used to non-invasively monitor the progression of disease. Dynamic lung compliance (FIG. 5B) and lung resistance (FIG. 5C) were measured at 6 and 11 days post-treatment. The specific anti-Pneumocystis antibody treatment had improved weight loss compared to control group. SSZ enhanced this effect. *p<0.05 for combination SSZ+Anti-Pneumocystis antibody treatment group compared to control treatment group at same time.

FIG. 6 is a graph showing that specific anti-Pc antibody treatment resulted in decreased lung Pc burden relative to control group at 11 days post-treatment. This effect is significantly enhanced with the addition of SSZ. Kexin is a single copy gene. *p<0.05 for combination SSZ+Anti-Pc Ab compared to control treatment, SSZ, or Anti-Pc Ab.

FIG. 7 is a graph showing Image Stream analysis which shows alveolar macrophages with internalized Pc. The specific anti-Pc antibody treatment group had increased alveolar macrophage internalization of Pc relative to control group, and this effect was significantly enhanced by the addition of SSZ.

FIG. 8 is a graph showing IFN-gamma ELISA analysis completed on BAL fluid. *p<0.05 for combination SSZ+Anti-Pc Ab treatment compared to control treatment, SSZ treatment, and Anti-Pc Ab treatment.

FIG. 9 is a graph comparing the percent weight loss of Pneumocystis infected mice on day 14 post-treatment with irrelevant antibody, IgM anti-Pneumocystis antibody, or IgG anti-Pneumocystis antibody.

FIG. 10 shows that treatment with antibodies to PCA1 accelerates resolution of lung damage in mice infected with Pneumocystis. Dynamic lung compliance was compared in Pneumocystis infected mice on day 14 post-treatment with irrelevant antibody, IgM anti-Pneumocystis antibody, or IgG anti-Pneumocystis antibody.

FIG. 11 is a graph comparing differential cell counts of Pneumocystis infected mice on day 14 post-treatment with irrelevant antibody, IgM anti-Pneumocystis antibody, or IgG anti-Pneumocystis antibody.

FIGS. 12A-12C show Pneumocystis infected mice treated with an IgM anti-Pneumocystis antibody pool or IgG anti-Pneumocystis antibody exhibit an increase in the M2 macrophage phenotype as compared to non-specific antibody. FIG. 12A is a graph that shows counts of the macrophage phenontype in Pneumocystis infected mice on day 14 post-treatment with irrelevant antibody, IgM anti-Pneumocystis antibody, or IgG anti-Pneumocystis antibody. Immunofluroescence was performed using antibodies to either iNOS (M1 macrophage marker) or YM-1 (M2 macrophage marker), and cells were imaged at 40× and counted. FIG. 12B is a graph that shows counts of the macrophage phenontype in Pneumocystis infected mice on day 14 post-treatment with irrelevant antibody, IgM anti-Pneumocystis antibody, or IgG anti-Pneumocystis antibody. Immunofluroescence was performed using antibodies to either iNOS (M1 macrophage marker) or arg-1 (M2 macrophage marker), and cells were imaged at 40× and counted. FIG. 12C shows representative immunofluorescence images of the counted cells.

### DETAILED DESCRIPTION OF THE INVENTION

The present invention relates to methods of treating or preventing Pneumocystis pneumonia infection, Pneumocystis cross-reactive antigen antigen 1 (“PCA1” or “Pca1”), and pharmaceutical compositions comprising PCA1.

One aspect of the present invention relates to a method of treating Pneumocystis pneumonia infection in a subject. This method involves administering to a subject having a Pneumocystis pneumonia infection one or more antibodies that bind specifically to a Pneumocystis cross-reactive antigen 1 (PCA1) protein under conditions effective to treat the Pneumocystis infection in the subject.

As used herein, Pneumocystis refers to Pneumocystis organisms derived from a variety of species, including mammals, such as mouse-, rat-, ferrit-, and human-derived Pneumocystis. The Pneumocystis cross-reactive antigen 1 (PCA1) may be derived from any mammal including, but not limited to, human, non-human primate, ungulate, rabbit, ferret, rat, or mouse.

In one embodiment, PCA1 is murine PCA1. The full-length mouse-derived PCA1 protein (GenBank Accession No. KX011348.1, which is hereby incorporated by reference in its entirety), identified herein as SEQ ID NO:1, is as follows:

```
Met Phe Phe Leu Arg Ile Ile Phe Ile Phe Ile Phe Leu Lys Ile Ser
1 5 10 15
Tyr Ala Glu Asn Thr Asp Lys Leu Ser Asp Phe Glu Lys Lys Tyr Pro
                        20 25 30
Glu Leu Tyr Gln Ala Asn Pro His Ala Leu Lys Leu Glu Ala Leu Lys
                35 40 45
Ser Gly Phe Ser Gly Lys Ser Val Lys Lys Gly Leu Gly Val Phe His
        50 55 60
Ile Gly Asn Leu Gly His Tyr Arg Asp His Lys Pro Val Ile Leu His
65 70 75 80
Val Ile Net Gly Leu Thr Val Gly Leu Ala Glu Cys Arg Gly Thr Leu
                                85 90 95
Ala Glu Arg Cys Lys Val Ile Lys Ala Leu Gly Asn Pro Ile Thr Gln
                        100 105 110
Tyr Cys Asn Lys Pro Tyr Asp Thr Cys Gln Asp Tyr Phe Asp Ala Arg
                115 120 125
Asn Tyr Leu Leu Pro Net Lys Asp Gln Leu Lys Asn Pro His Ala His
        130 135 140
His Asp Ala Cys Arg Thr Ile Leu Leu Asn Cys Leu Phe Phe Lys His
145 150 155 160
Arg Asn Tyr Ile Thr Ser Asp Cys Val Pro Leu Val Ala Leu Cys Tyr
                                165 170 175
Leu Arg Val Arg Gln Asn Phe Val Glu Ala Ile Net Thr Glu Ala Leu
                        180 185 190
Arg Gly Glu Ile Asn Thr Lys Gly Ala Ala Ala Ala Net Lys Lys Val
                195 200 205
Cys Glu Lys Ile Gly His Glu Ser Pro Asp Leu Leu His Leu Cys Phe
        210 215 220
Lys Thr Thr Val Leu Glu Lys Pro Lys Arg Ser Asn Lys Gln Tyr Ile
225 230 235 240
Glu Asp Val Lys Ser Arg Ile Arg Thr Val Ser Thr Gly Asn Cys Arg
                                245 250 255
Gln Val Leu Glu Glu Cys Tyr Phe Asn Val Leu Asp Tyr Pro Asp Ile
                        260 265 270
Tyr Gln Ser Cys Arg Asn Phe Arg Arg Phe Cys Ser Glu Ile Gly Val
                275 280 285
Val Tyr Thr Pro Val Asp Ser Thr Phe Asp Leu Phe Gln Lys Pro Leu
        290 295 300
Ser Ala Glu Lys Leu Leu Ile Asp Thr Ser Ser Lys Ile Ser Glu Asp
305 310 315 320
Leu Gly Leu Gly Phe Ser Lys Tyr Val Gln Lys Lys Ser Ser Asn Leu
                                325 330 335
Glu Ile Ala Ala Tyr Leu Val Asn Lys Thr Trp Val Tyr Asp Asn Asp
                        340 345 350
Cys Arg Asn Lys Leu Lys Glu Leu Cys Leu His Ile Ala Ser Leu Pro
                355 360 365
Leu Thr Lys Gln Leu Cys Thr Leu Ala His Asp Arg Asn Ser Lys Leu
        370 375 380
Cys Arg Asp Phe Tyr Asn Ser Ile Gly Thr Glu Cys Tyr Ser Leu Tyr
385 390 395 400
Tyr Glu Phe Lys Asn Val Gly Leu Leu Tyr Asn Tyr Thr Tyr Arg Leu
                                405 410 415
Ser Arg Asp Gln Cys Ser Lys Tyr Val Glu Arg Cys Leu Phe Leu Arg
                        420 425 430
Glu Gln Tyr Ala Tyr Trp Asn Ser Leu Asp Thr Cys Ala Asn Val Phe
                435 440 445
Ser Ser Cys Tyr Lys Glu Asp Net Asp Phe Ser Ala Lys Leu Asp Leu
        450 455 460
Leu Asn Arg Ile Lys Asp Lys Ile Val Val Pro Lys Gly Asn Thr Arg
465 470 475 480
Tyr Phe Val Glu Leu Leu Cys Lys Ser Tyr Ile Val Ala Glu Cys Ser
                                485 490 495
Ala Ser Asp Leu Net Phe Lys Ser Tyr Ala Leu Net Glu Ala Cys Leu
                        500 505 510
His Pro Glu Arg Ile Cys Arg Glu Leu Lys Asn His Phe Ser Glu Glu
                515 520 525
Ser Arg Lys Leu Glu Asn Lys Leu Arg Ser Ile Leu Lys Pro Thr Tyr
        530 535 540
Tyr Glu Cys Lys Asp Leu Gly Gln Lys Cys Asn Ser Gly Phe Tyr Ph
545 550 555 560
Asp Gly Asp Ile Glu Ala Gln Cys Asn His Phe Lys Lys Arg Cys Gln
                                565 570 575
Asp Lys Gln Glu Arg Leu Lys Leu Ile Asn His Ile Val Asp Ser Ser
                        580 585 590
Ala Leu Tyr Leu Ala Asn Glu Val Gln Cys Arg Thr Tyr Phe Asp Ser
                595 600 605
Phe Cys Gly Ala Asn Val Lys Gln Glu Phe Lys Gln Ile Cys Asn Lys
        610 615 620
Gly Ala Asn Gly Ile Cys Pro Asp Ile Ile Asp Asp Ser Lys Glu His
625 630 635 640
Cys Ala His Leu Ile Asn His Leu Thr Ser Leu Gly Ile Ser Ser Ser
                                645 650 655
Ser Ala Ser Leu Pro Leu Asp Tyr Cys Asp Ser Ala Ile Asn Tyr Cys
                        660 665 670
Asn Ser Leu Ser Lys Phe Cys Thr Glu Ser Lys Arg Gln Cys Asp Ser
                675 680 685
Val Ile Ser Phe Cys Thr Ser Glu Ser Lys Lys Thr Asp Glu Tyr Gly
        690 695 700
Ser Phe Ile Asp Gln Tyr Pro Ala Ala Ala Ala Asn Ala Thr Lys Cys
705 710 715 720
Lys Val Thr Leu Lys Glu Leu Cys Gln Asp Ser Ser Lys Lys Asp Ser
                                725 730 735
Tyr Ser Thr Leu Cys Ala Tyr Asn Lys Asp Gly Tyr Thr Glu Ile Cys
                        740 745 750
Lys Asn Leu Arg Asn Phe Ile Glu Lys Ala Cys Glu Asn Leu Arg Ile
                755 760 765
His Leu His Thr Tyr Asp Thr Asn Ser Leu Asn Thr Asn Lys Gly Ser
        770 775 780
Ala Gln Asp Arg Cys Thr Tyr Ile Arg Asn Leu Tyr Phe Lys Phe Lys
785 790 795 800
Asn Ile Cys Leu Leu Val Asp Pro Phe Tyr Asp Leu Ser Pro Ile Ile
                                805 810 815
Thr Gln Glu Cys Lys Thr Asn Ile Ser Glu Pro Ala Leu Pro Asp Lys
                        820 825 830
Asp Pro Gln Pro Thr Ser Ser Pro Gln Pro Lys Pro Arg Pro Arg Pro
                835 840 845
Arg Pro Gln Pro Gln Pro His Pro His Pro Lys Pro Gln Pro Gln Pro
        850 855 860
Thr Pro Glu Pro Gln Pro Gln Pro Ala Pro Glu Pro Arg Pro Gln Pro
865 870 875 880
Thr Ser Lys Pro Arg Pro Gln Pro Thr Ser Lys Pro Arg Pro Gln Pro
                                885 890 895
Thr Pro Glu Pro Arg Pro Leu Pro Val Pro Gly Pro Gly Pro Leu Pro
                        900 905 910
Val Pro Gly Pro Arg Pro Gln Pro Gln Pro Gln Pro Gln Pro Gln Pro
                915 920 925
Gln Pro Gln Pro Gln Pro Gln Pro Gln Pro Gln Pro Gln Pro Gln Pro
        930 935 940
Gln Pro Gln Pro Gln Pro Gln Pro Lys Pro Gln Pro Pro Ser Gln Ser
945 950 955 960
Thr Ser Glu Ser Ala Ser Gln Ser Lys Pro Lys Pro Thr Thr Gln Thr
                                965 970 975
Lys Pro Ser Pro Arg Pro His Pro Lys Pro Val Pro Lys Pro Ser Ser
                        980 985 990
Ile Asp Thr Gly Pro Ser Lys Ser Asp Ser Ser Phe Ile Phe Thr Val
                995 1000 1005
Thr Lys Thr Ile Thr Lys Ile Ser Glu Thr Glu Lys Pro Ser Thr
        1010 1015 1020
Lys Pro Ser Val Lys Pro Thr Ser Thr Lys Thr Thr Ser Lys Pro
        1025 1030 1035
Ser Thr Lys Pro Ser Thr Lys Pro Ser Val Lys Pro Ala Ser Thr
        1040 1045 1050
Lys Thr Thr Ser Glu Ser Glu Lys Pro Thr Leu Glu Glu Val Pro
        1055 1060 1065
Glu Thr Lys Gly Asn Gly Val Arg Val Ile Gly Phe Glu Gly Leu
        1070 1075 1080
Gln Leu Leu Ser Net Ile Val Ala Ile Ile Ile Gly Ile Trp Ile Met
        1085 1090 1095
```

As noted supra, PCA1 may be derived from any mammal including, but not limited to, human, non-human primate, ungulate, rabbit, ferret, rat, or mouse.

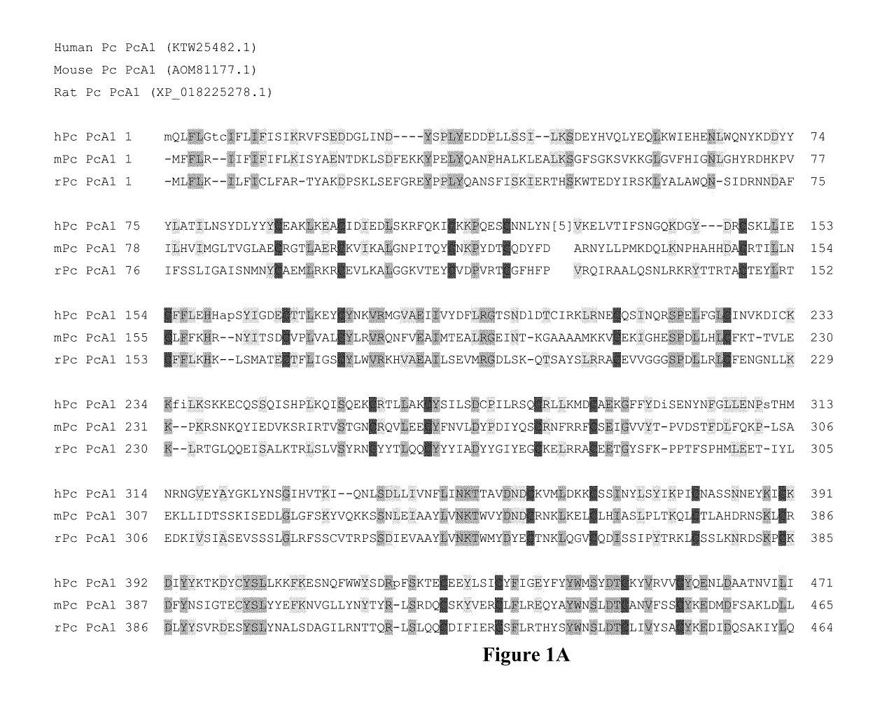

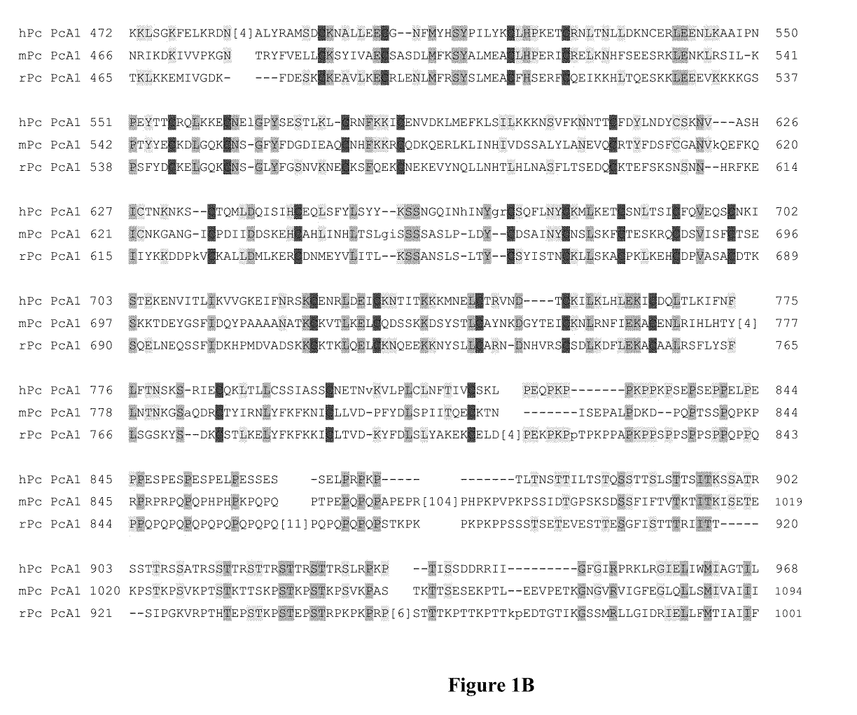

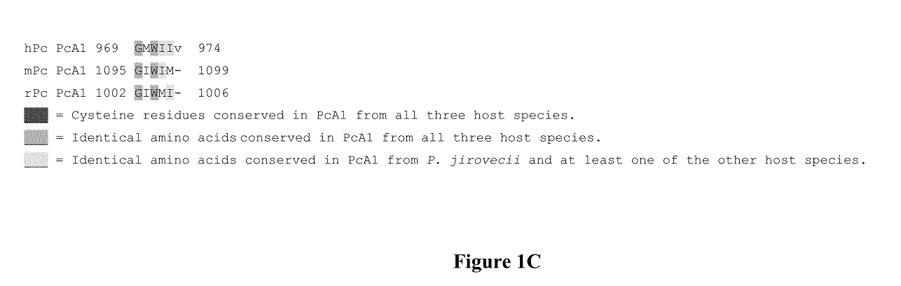

While the PCA1 amino acid sequences infecting different species are themselves quite different between species, the proteins share a conserved cysteine backbone (FIGS. 1A-1C).

In carrying out this method of the present invention, a subject having a Pneumocystis pneumonia infection is administered one or more antibodies that bind specifically to a Pneumocystis cross-reactive antigen 1 (PCA1) protein.

Antibodies that bind specifically to a PCA1 protein or polypeptide can be monoclonal antibodies, polyclonal antibodies, or functional fragments or variants thereof.

As described in the Examples (infra), such antibodies that bind to PCA1 should also be “cross-reactive” antibodies. By “cross-reactive” it is meant that an antibody generated from one species of PCA1 will bind PCA1 from that species and other species. For example, a cross-reactive antibody that binds specifically to murine PCA1 will also bind to PCA1 from human.

Three cross-reactive monoclonal anti-Pneumocystis antibodies—4F11, 1G4, and 5E12—have been previously described (Gigliotti and Harmsen, “Pneumocystis carinii Host Origin Defines the Antibody Specificity and Protective Response Induced by Immunization,” J. Infect. Dis. 176(5):1322-6 (1997); Gigliotti et al., “Development of Murine Monoclonal Antibodies to Pneumocystis carinii,” J. Infect. Dis. 154(2):315-22 (1986), which are hereby incorporated by reference in their entirety). The advantage of a cross-reactive antibody is that if PCA1 induces antibody that is cross-reactive with pneumocystis from a range of mammalian hosts, it is likely that PCA1 would induce an immune response broadly reactive with most, if not all, human pneumocystis.

In one embodiment, the antibody administered pursuant to this and other methods of the present invention binds to one or more epitopes in SEQ ID NO: 1. The epitope may comprise about 8, 9, 10, 11, 12, 13, 14, 15, 16, 17, 18, 19, 20, 21, 22, 23, 24, 25, or more amino acids.

In another embodiment, the antibody administered pursuant to this and other methods of the present invention binds to one or more epitopes in PCA1 from a species other than murine. PCA1 from a species other than mouse may have a low sequence identity to PCA1 of SEQ ID NO:1. However, the PCA1 protein or polypeptide from another species will have a conserved cysteine backbone, such that at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cysteine residues in the PCA1 polypeptide are conserved or match the murine PCA1 protein or polypeptide.

In carrying out this method of the present invention, the method may further comprise repeating said initial administering. Repeating administering may be carried out twice or numerous times, as needed, to treat the subject.

In one embodiment, administering an antibody is carried out orally, by inhalation, by intranasal instillation, topically, transdermally, parenterally, subcutaneously, by intravenous injection, by intra-arterial injection, by intramuscular injection, intraplurally, intraperitoneally, by intracavitary or intravesical instillation, intraocularly, intraventricularly, intralesionally, intraspinally, or by application to mucous membranes.

In a further embodiment, the antibody administered can be a humanized antibody. According to one specific embodiment, the humanized antibody is also active in mouse. The potential advantage of this is that a potential human vaccine could be tested in animal (mouse) models and, if warranted, based on model results, be moved to clinical trial with supporting data for its activity in humans (Tesini et al., “Immunization With Pneumocystis Cross-Reactive Antigen 1 (Pca1) Protects Mice Against Pneumocystis Pneumonia and Generates Antibody to Pneumocystis jirovecii,” Infection and Immunity 85(4):e00850-16 (2017), which is hereby incorporated by reference in its entirety).

Monoclonal antibody production can be effected by techniques that are well-known in the art. Basically, the process involves first obtaining immune cells (lymphocytes) from the spleen of a mammal (e.g., mouse) that has been previously immunized with the antigen of interest (e.g., PCA1 protein or a polypeptide fragment thereof or an immunogenic conjugate) either in vivo or in vitro. The antibody-secreting lymphocytes are then fused with myeloma cells or transformed cells, which are capable of replicating indefinitely in cell culture. The resulting fused cells, or hybridomas, are immortal, immunoglobulin-secreting cell lines that can be cultured in vitro. Upon culturing the hybridomas, the resulting colonies can be screened for the production of desired monoclonal antibodies. Colonies producing such antibodies are cloned and grown either in vivo or in vitro to produce large quantities of antibody. A description of the theoretical basis and practical methodology of fusing such cells is set forth in Kohler and Milstein, “Continuous Cultures of Fused Cells Secreting Antibody of Predefined Specificity,” Nature 256:495 (1975), which is hereby incorporated by reference in its entirety.

Mammalian lymphocytes are immunized by in vivo immunization of the animal (e.g., mouse, rat, rabbit, or human) with the protein or polypeptide or immunogenic conjugates of the invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, “Derivation of Specific Antibody-Producing Tissue Culture and Tumor Lines by Cell Fusion,” Eur. J. Immunol. 6:511 (1976), which is hereby incorporated by reference in its entirety). This immortal cell line, which is preferably murine, but may also be derived from cells of other mammalian species including, but not limited to, rats and humans, is selected to be deficient in enzymes necessary for the utilization of certain nutrients, to be capable of rapid growth, and to have good fusion capability. Many such cell lines are known to those skilled in the art, and others are regularly described. Human hybridomas can be prepared using the EBV-hybridoma technique monoclonal antibodies (Cole et al., in Monoclonal Antibodies and Cancer Therapy, Alan R. Liss, Inc., pp. 77-96 (1985), which is hereby incorporated by reference in its entirety). Human antibodies may be used and can be obtained by using human hybridomas (Cote et al., “Generation of Human Monoclonal Antibodies Reactive with Cellular Antigens,” Proc. Natl. Acad. Sci. USA 80:2026-2030 (1983), which is hereby incorporated by reference in its entirety) or by transforming human B cells with EBV virus in vitro (Cole et al., in Monoclonal Antibodies and Cancer Therapy, Alan R. Liss, Inc., pp. 77-96 (1985), which is hereby incorporated by reference in its entirety). In addition, monoclonal antibodies can be produced in germ-free animals (see PCT/US90/02545, which is hereby incorporated by reference in its entirety).

Procedures for raising polyclonal antibodies are also well known. Typically, such antibodies can be raised by administering the antigen (the protein or polypeptide or immunogenic conjugates of the invention) subcutaneously to rabbits, mice, or rats which have first been bled to obtain pre-immune serum. The antigens can be injected as tolerated. Each injected material can contain adjuvants and the selected antigen (preferably in substantially pure or isolated form). Suitable adjuvants include, without limitation, Freund's complete or incomplete mineral gels such as aluminum hydroxide, surface active substances such as lysolecithin, pluronic polyols, polyanions, peptides, oil emulsions, dinitrophenol, and potentially useful human adjuvants such as bacille Calmette-Guerin and Carynebacterium parvum. The subject mammals are then bled one to two weeks after the first injection and periodically boosted with the same antigen (e.g., three times every six weeks). A sample of serum is then collected one to two weeks after each boost. Polyclonal antibodies can be recovered from the serum by affinity chromatography using the corresponding antigen to capture the antibody. This and other procedures for raising polyclonal antibodies are disclosed in Harlow & Lane, eds., Antibodies: A Laboratory Manual (1988), which is hereby incorporated by reference in its entirety.

In addition, techniques developed for the production of chimeric antibodies (Morrison et al., “Chimeric Human Antibody Molecules: Mouse Antigen-Binding Domains with Human Constant Region Domains,” Proc. Natl. Acad. Sci. USA 81:6851-6855 (1984); Neuberger et al., “Recombinant Antibodies Possessing Novel Effector Functions,” Nature 312:604-608 (1984); Takeda et al., “Construction of Chimeric Processed Immunoglobulin Gene Containing Mouse Variable and Human Constant Region Sequences,” Nature 314:452-454 (1985), each of which is hereby incorporated by reference in its entirety) by splicing the genes from a mouse antibody molecule of appropriate antigen specificity together with genes from a human antibody molecule of appropriate biological activity can be used. For example, the genes from a mouse antibody molecule specific for epitopes in PCA1 protein or polypeptide of the present invention can be spliced together with genes from a human antibody molecule of appropriate biological activity. A chimeric antibody is a molecule in which different portions are derived from different animal species, such as those having a variable region derived from a murine mAb and a human immunoglobulin constant region (e.g., U.S. Pat. No. 4,816,567 to Cabilly et al. and U.S. Pat. No. 4,816,397 to Boss et al., each of which is hereby incorporated by reference in its entirety).

In addition, techniques have been developed for the production of humanized antibodies (e.g., U.S. Pat. No. 5,585,089 to Queen and U.S. Pat. No. 5,225,539 to Winter, each of which is hereby incorporated by reference in its entirety). An immunoglobulin light or heavy chain variable region includes a “framework” region interrupted by three hypervariable regions, referred to as complementarity determining regions (CDRs). The extent of the framework region and CDRs have been precisely defined (see Kabat et al., “Sequences of Proteins of Immunological Interest,” U.S. Department of Health and Human Services (1983), which is hereby incorporated by reference in its entirety). Briefly, humanized antibodies are antibody molecules from non-human species having one or more CDRs from the non-human species and a framework region from a human immunoglobulin molecule.

Alternatively, techniques described for the production of single chain antibodies (e.g., U.S. Pat. No. 4,946,778 to Ladner et al.; Bird, “Single-Chain Antigen Binding Protein,” Science 242:423-426 (1988); Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Ward et al., “Binding Activities of a Repertoire of Single Immunoglobulin Variable Domains Secreted from Escherichia coli,” Nature 334:544-546 (1989), each of which is hereby incorporated by reference in its entirety) can be adapted to produce single chain antibodies against a PCA1 protein or polypeptide of the present invention. Single chain antibodies are formed by linking the heavy and light chain fragments of the Fv region via an amino acid bridge, resulting in a single chain polypeptide.

In addition to utilizing whole antibodies, the present invention also encompasses use of binding portions of such antibodies. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, Academic Press (New York), pp. 98-118 (1983), which is hereby incorporated by reference in its entirety. Alternatively, the Fab fragments can be generated by treating the antibody molecule with papain and a reducing agent. Alternatively, Fab expression libraries may be constructed (Huse et al., “Generation of a Large Combinatorial Library of the Immunoglobulin Repertoire in Phage Lambda,” Science 246:1275-1281 (1989), which is hereby incorporated by reference in its entirety) to allow rapid and easy identification of monoclonal Fab fragments with the desired specificity.

Antibodies of the present invention may be isolated by standard techniques known in the art, such as immunoaffinity chromatography, centrifugation, precipitation, etc. The antibodies (or fragments or variants thereof) are preferably prepared in a substantially purified form (i.e., at least about 85% pure, 90% pure, 95% to 99% pure).

From the foregoing, it should be appreciated that the present invention also relates to the isolated immune sera containing the polyclonal antibodies, compositions containing monoclonal antibodies, or fragments or variants thereof.

In addition, antibodies generated can also be used in the production of anti-idiotypic antibody. The anti-idiotypic antibody can then in turn be used for immunization, in order to produce a subpopulation of antibodies that bind the initial antigen of the pathogenic microorganism, e.g., epitopes on PCA1 protein or polypeptide of the present invention (Jerne, “Towards a Network Theory of the Immune System,” Ann. Immunol. (Paris) 125c:373 (1974); Jerne et al., “Recurrent Idiotypes and Internal Images,” EMBO J. 1:234 (1982), each of which is hereby incorporated by reference in its entirety).

Another type of active agent is an expression vector encoding an immunogenic protein or polypeptide (or fusion protein), which expression vector can be used for in vivo expression of the protein or polypeptide (PCA1) in eukaryotic, preferably mammalian, organisms. Hence, this aspect relates to a DNA vaccine.

DNA inoculation represents a relatively new approach to vaccine and immune therapeutic development. The direct injection of gene expression cassettes (i.e., as plasmids) into a living host transforms a number of cells into factories for production of the introduced gene products. Expression of these delivered genes has important immunological consequences and can result in the specific immune activation of the host against the novel expressed antigens. This approach to immunization can overcome deficits of traditional antigen-based approaches and provide safe and effective prophylactic and therapeutic vaccines. The transfected host cells can express and present the antigens to the immune system (i.e., by displaying fragments of the antigens on their cell surfaces together with class I or class II major hisotcompatibility complexes). DNA vaccines recently have been shown to be a promising approach for immunization against a variety of infectious diseases (Michel et al., “DNA-Mediated Immunization to the Hepatitis B Surface Antigen in Mice: Aspects of the Humoral Response Mimic Hepatitis B Viral Infection in Humans,” Proc. Nat'l Acad. Sci. USA 92:5307-5311 (1995), which is hereby incorporated by reference in its entirety). Delivery of naked DNAs containing microbial antigen genes can induce antigen-specific immune responses in the host. The induction of antigen-specific immune responses using DNA-based vaccines has shown some promising effects (Wolff et al., “Long-Term Persistence of Plasmid DNA and Foreign Gene Expression in Mouse Muscle,” Hum. Mol. Genet. 1:363-369 (1992), which is hereby incorporated by reference in its entirety).

The DNA vaccine can also be administered together with a protein-based vaccine, either as a single formulation or two simultaneously introduced formulations. See PCT Publication No. WO 2008/082719 to Rose et al., which is hereby incorporated by reference in its entirety.

According to one approach, the expression vector (to be used as a DNA vaccine) is a plasmid containing a DNA construct encoding the PCA1 protein or polypeptide of the present invention. The plasmid DNA can be introduced into the organism to be exposed to the DNA vaccine, preferably via intramuscular or dermal injection, which plasmid DNA can be taken up by muscle or dermal cells for expression of the PCA1 protein or polypeptide of the present invention.

According to another approach, the expression vector (to be used as a DNA vaccine) is an infective transformation vector, such as a viral vector.

When an infective transformation vector is employed to express a PCA1 protein or polypeptide of the present invention in a host organism's cell, conventional recombinant techniques can be employed to prepare a DNA construct that encodes the protein or polypeptide and ligate the same into the infective transformation vector (Sambrook et al., Molecular Cloning: A Laboratory Manual, Second Edition, Cold Spring Harbor Press, NY (1989), which is hereby incorporated by reference in its entirety). The infective transformation vector so prepared can be maintained ex vivo in appropriate host cell lines, which may include bacteria, yeast, mammalian cells, insect cells, plant cells, etc. For example, having identified the protein or polypeptide to be expressed in cells of a host organism, a DNA molecule that encodes the oligoRNA can be ligated to appropriate 5′ promoter regions and 3′ transcription termination regions, forming a DNA construct, so that the protein or polypeptide will be appropriately expressed in transformed cells. The selection of appropriate 5′ promoters and 3′ transcription termination regions is well known in the art and can be performed with routine skill. Suitable promoters for use in mammalian cells include those identified herein.

Any suitable viral vector can be utilized to express the PCA1 protein or polypeptide of the present invention. When transforming mammalian cells for heterologous expression of a PCA1 protein or polypeptide of the present invention, exemplary viral vectors include adenovirus vectors, adeno-associated vectors, and retroviral vectors. Other suitable viral vectors now known or hereafter developed can also be utilized to deliver into cells a DNA construct encoding a protein or polypeptide of the present invention.

Adenovirus gene delivery vehicles can be readily prepared and utilized given the disclosure provided in Berkner, “Development of Adenovirus Vectors for the Expression of Heterologous Genes,” Biotechniques 6:616-627 (1988); Rosenfeld et al., “Adenovirus-Mediated Transfer of a Recombinant α1-Antitrypsin Gene to the Lung Epithelium In Vivo,” Science 252:431-434 (1991); and PCT Publication Nos. WO 93/07283, WO 93/06223, and WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curie′, each of which is hereby incorporated by reference in its entirety.

Adeno-associated viral gene delivery vehicles can be constructed and used to deliver into cells a DNA construct encoding a PCA1 protein or polypeptide of the present invention. The use of adeno-associated viral gene delivery vehicles in vitro is described in Chatterjee et al., “Dual Target Inhibition of HIV-1 In Vitro by Means of an Adeno-Associated Virus Antisense Vector,” Science 258:1485-1488 (1992); Walsh et al., “Phenotypic Correction of Fanconi Anemia In Human Hematopoietic Cells with Recombinant Adeno-Associated Virus Vector,” J. Clin. Invest. 94:1440-1448 (1994); Flotte et al., “Expression of the Cystic Fibrosis Trnasmembrane Conductance Regulator from a Novel Adeno-Associated Virus Promoter,” J. Biol. Chem. 268:3781-3790 (1993), each of which is hereby incorporated by reference in its entirety.

Retroviral vectors which have been modified to form infective transformation systems can also be used to deliver into cells a DNA construct encoding a PCA1 protein or polypeptide of the present invention. One such type of retroviral vector is disclosed in U.S. Pat. No. 5,849,586 to Kriegler et al., which is hereby incorporated by reference in its entirety.

Alternatively, a colloidal dispersion system can be used to deliver the DNA vaccine to the subject. Colloidal dispersion systems include macromolecule complexes, nanocapsules, microspheres, beads, and lipid-based systems including oil-in-water emulsions, micelles, mixed micelles, and liposomes. In one embodiment, the colloidal system is a lipid preparation including unilamaller and multilamellar liposomes.

Liposomes are artificial membrane vesicles that are useful as delivery vehicles in vitro and in vivo. It has been shown that large unilamellar vesicles (LUV), which range in size from about 0.2 to about 4.0 μm, can encapsulate a substantial percentage of an aqueous buffer containing DNA molecules (Fraley et al., “New Generation Liposomes: The Engineering of an Efficient Vehicle for Intracellular Delivery of Nucleic Acids,” Trends Biochem. Sci. 6:77 (1981), which is hereby incorporated by reference in its entirety). In addition to mammalian cells, liposomes have been used for delivery of polynucleotides in yeast and bacterial cells. For a liposome to be an efficient transfer vehicle, the following characteristics should be present: (1) encapsulation of the DNA molecules at high efficiency while not compromising their biological activity; (2) substantial binding to host organism cells; (3) delivery of the aqueous contents of the vesicle to the cell cytoplasm at high efficiency; and (4) accurate and effective expression of genetic information (Mannino et al., “Liposome Mediated Gene Transfer,” Biotechniques 6:682-90 (1988), which is hereby incorporated by reference in its entirety). In addition to such LUV structures, multilamellar and small unilamellar lipid preparations, which incorporate various cationic lipid amphiphiles can also be mixed with anionic DNA molecules to form liposomes (Feigner et al., “Lipofection: A highly Efficient, Lipid-Mediated DNA-Transfection Procedure,” Proc. Natl. Acad. Sci. USA 84(21): 7413-7 (1987), which is hereby incorporated by reference in its entirety).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and typically the presence of divalent cations. The appropriate composition and preparation of cationic lipid amphiphile:DNA formulations are known to those skilled in the art, and a number of references which provide this information are available (e.g., Bennett et al., “Considerations for the Design of Improved Cationic Amphiphile-Based Transfection Reagents,” J. Liposome Research 6(3):545 (1996), which is hereby incorporated by reference in its entirety).

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine, and distearoylphosphatidylcholine. Examples of cationic amphiphilic lipids useful in formulation of nucleolipid particles for polynucleotide delivery include the monovalent lipids N-[1-(2,3-dioleoyloxy)propyl]-N,N,N,-trimethyl ammonium methyl-sulfate, N-[2,3-dioleoyloxy)propyl]-N,N,N-trimethyl ammonium chloride, and DC-cholesterol, the polyvalent lipids LipofectAMINE™, dioctadecylamidoglycyl spermine, Transfectam®, and other amphiphilic polyamines. These agents may be prepared with helper lipids such as dioleoyl phosphatidyl ethanolamine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization. The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further alternative for delivery of DNA is the use of a polymeric matrix which can provide either rapid or sustained release of the DNA vaccine to the organism. A number of polymeric matrices are known in the art and can be optimized with no more than routine skill.

Passive immunotherapy with antibody preparations have been used successfully in many infectious diseases. Because of the immunocompromised host's altered ability to respond to active immunization, passive immunotherapy is a way to provide the benefit of antibody without the necessity of a specific immune response in the recipient. While often used to prevent diseases, e.g., varicella immune globulin in the compromised host, it can be used therapeutically. The use of immunoglobulin has been shown to improve the outcome of CMV disease, particularly pneumonitis, and enteroviral encephalitis, in the immunocompromised human host (Ljungman, “Cytomegalovirus Pneumonia: Presentation, Diagnosis, and Treatment,” Semin. Respir. Infect. 10(4):209-215 (1995); Dwyer et al., “Intraventricular Gamma-globulin for the Management of Enterovirus Encephalitis,” Pediatr. Infect. Dis. J. 7(5 Suppl): S30-3 (1988), each of which is hereby incorporated by reference in its entirety). Animal models support this approach in a variety of fungal infections (Casadevall et al., “Return to the Past: The Case for Antibody-based Therapies in Infectious Diseases,” Clin. Infect. Dis. 21(1):150-161 (1995), which is hereby incorporated by reference in its entirety).

In one embodiment of this and the other methods of the present invention, the one or more antibodies bind to one or more epitopes in SEQ ID NO:1 or a PCA1 protein or polypeptide from a species other than mouse.

In another embodiment of this and the other methods of the present invention, the one or more antibodies comprise monoclonal antibody 4F11.

In a further embodiment of this and the other methods of the present invention, the one or more antibodies comprise monoclonal antibody 1G4.

In yet another embodiment of this and the other methods of the present invention, an antibiotic agent effective against Pneumocystis and/or an immunomodulatory agent is administered along with the one or more antibodies that bind specifically to a Pneumocystis cross-reactive antigen 1 (PCA1).

In another embodiment, in carrying out this and other methods of the present invention, the method further comprises administering to the subject an antibiotic agent effective against Pneumocystis and/or immunomodulatory agent. Suitable antibiotic agents are include, without limitation, co-trimoxazole, pentamidine, primaquine, and trimethoprim plus sulfamethoxazole.

The term “immunomodulatory agent” means a pharmaceutically active compound that enhances, suppresses, or otherwise affects a subject's immune system.

In one embodiment, the immunomodulatory agent is an anti-inflammatory. Anti-inflammatories include any of a number of compounds, agents, therapeutic mediums or drugs known to those skilled in the art, either steroidal or non-steroidal, and generally characterized as having the property of counteracting or suppressing the inflammatory process. One exemplary anti-inflammatory includes, without limitation, sulfasalazine.

In this and other methods of the present invention, the subject to be treated is preferably a mammal. Exemplary mammals to be treated include, without limitation, ungulates, non-human primates, rabbits, ferrets, rats, and mice.

In one embodiment, this method of the present invention involves further administering to the subject a Pneumocystis protein or polypeptide comprising the amino acid sequence of SEQ ID NO:1, or other related PCA1 protein or polypeptide, or antigenic fragment thereof.

The PCA1 protein or polypeptide of the present invention can be used to induce active immunity against Pneumocystis. Thus, another aspect of the present invention relates to treating a subject at risk for Pneumocystis pneumonia infection. This method involves administering to a subject at risk for Pneumocystis pneumonia infection a Pneumocystis protein or polypeptide comprising the amino acid sequence of SEQ ID NO:1 under conditions effective to prevent the Pneumocystis infection.

In one embodiment, a subject at risk for Pneumocystis pneumonia infection includes, but is not limited to, a subject having a compromised immune system or a subject having a malfunction of antibodies and/or CD4⁺ cells relative to a normally healthy individual of the same health category (e.g., age, race, sex, family history, etc.).

In another embodiment, the subject administered the Pneumocystis protein or polypeptide comprising the amino acid sequence of SEQ ID NO:1 or related protein or polypetide may further be administered a booster of the protein or polypeptide under conditions effective to enhance immunization of the subject.

The PCA1 protein or polypeptide of the present invention can be administered to a subject orally, by inhalation, by intranasal instillation, topically, transdermally, parenterally, subcutaneously, by intravenous injection, by intra-arterial injection, by intramuscular injection, intraplurally, intraperitoneally, by intracavitary or intravesical instillation, intraocularly, intraventricularly, intralesionally, intraspinally, or by application to mucous membranes.

The PCA1 protein or polypeptide may be administered alone or with suitable pharmaceutical carriers, and can be in solid or liquid form such as, tablets, capsules, powders, solutions, suspensions, or emulsions.

Use of active immunization in an immunocompromised host would seem counter-intuitive. However, the use of vaccines in immunocompromised humans has been extensively reviewed by Pirofski and Casadevall, “Use of Licensed Vaccines for Active Immunization of the Immunocompromised Host,” Clin. Microbiol. Rev. 11(1):1-26 (1998), which is hereby incorporated by reference in its entirety. Clinical trials have demonstrated the immunogenicity of H. influenzae vaccines in children with cancer and sickle cell disease (Feldman et al., “Risk of Haemophilus influenzae Type b Disease in Children with Cancer and Response of Immunocompromised Leukemic Children to a Conjugate Vaccine,” J. Infect. Dis. 161(5):926-931 (1990); Shenep et. al., “Response of Immunocompromised Children with Solid Tumors to a Conjugate Vaccine for Haemophilus influenzae Type b,” J. Pediatr. 125(4):581-584 (1994); Gigliotti et al., “Immunization of Young Infants with Sickle Cell Disease with a Haemophilus influenzae Type b Saccharide-Diphtheria CRM197 Protein Conjugate Vaccine,” J. Pediatr. 114(6):1006-10 (1989); Gigliotti et al., “Serologic Follow-up of Children With Sickle Cell Disease Immunized with a Haemophilus influenzae Type b Conjugate Vaccine During Early Infancy,” J. Pediatr. 118(6):917-919 (1991), each of which is hereby incorporated by reference in its entirety). New developments in vaccine technology should enhance the ability to vaccinate at-risk hosts.

In each of the embodiments that involves the induction of active immunity, immunostimulants may be co-administered to increase the immunological response. The term “immunostimulant” is intended to encompass any compound or composition which has the ability to enhance the activity of the immune system, whether it be a specific potentiating effect in combination with a specific antigen, or simply an independent effect upon the activity of one or more elements of the immune response. Immunostimulant compounds include but are not limited to mineral gels, e.g., aluminum hydroxide; surface active substances such as lysolecithin and pluronic polyols; polyanions; peptides; oil emulsions; alum; and MDP. Methods of utilizing these materials are known in the art, and it is well within the ability of the skilled artisan to determine an optimum amount of immunostimulant for a given active vaccine. More than one immunostimulant may be used in a given formulation. The immunogen may also be incorporated into liposomes, or conjugated to polysaccharides and/or other polymers for use in a vaccine formulation.

In one embodiment, an antibiotic agent effective against Pneumocystis is administered along with the Pneumocystis protein or polypeptide comprising the amino acid sequence of SEQ ID NO:1. Suitable antibiotic agents include those selected from the group consisting of co-trimoxazole, pentamidine, primaquine, and trimethoprim plus sulfamethoxazole.

In another embodiment, the prevention of infection by these organisms can be carried out by also administering to a patient one or more antibodies that bind specifically to a PCA1 protein of the present invention along with the Pneumocystis protein or polypeptide comprising the amino acid sequence of SEQ ID NO:1.

Regardless of the method of the present invention to be employed, i.e., either passive or active immunity, the immunopotency of a composition can be determined by monitoring the immune response of test animals following their immunization with the composition. Monitoring of the immune response can be conducted using any immunoassay known in the art. Generation of a humoral (antibody) response and/or cell-mediated immunity may be taken as an indication of an immune response. Test animals may include mice, hamsters, dogs, cats, monkeys, rabbits, chimpanzees, etc., and eventually human subjects.

The immune response of the test subjects can be analyzed by various approaches such as: the reactivity of the resultant immune serum to the PCA1 protein or polypeptide, as assayed by known techniques, e.g., enzyme linked immunosorbent assay (“ELISA”), immunoblots, immunoprecipitations, etc.; or, by protection of immunized hosts from infection by the pathogen and/or attenuation of symptoms due to infection by the pathogen in immunized hosts as determined by any method known in the art, for assaying the levels of an infectious disease agent, e.g., the bacterial levels (e.g., by culturing of a sample from the patient), etc. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

After vaccination of an animal using the methods and compositions of the present invention, any binding assay known in the art can be used to assess the binding between the resulting antibody and the particular molecule. These assays may also be performed to select antibodies that exhibit a higher affinity or specificity for the particular antigen.

The antibodies or binding portions of the present invention are also useful for detecting in a sample the presence of epitopes of PCA1 protein or polypeptide of the present invention and, therefore, the presence of either proteins containing the epitopes of PCA1 protein or polypeptide, as well as Pneumocystis. This detection method includes the steps of providing an isolated antibody or binding portion thereof raised against an epitope containing PCA1 protein or polypeptide of the present invention, adding to the isolated antibody or binding portion thereof a sample suspected of containing a quantity of PCA1 protein or polypeptide or whole Pneumocystis, and then detecting the presence of a complex comprising the isolated antibody or binding portion thereof bound to the epitope (or protein or polypeptide or whole organism, as noted above).

Immunoglobulins, particularly antibodies (and functionally active fragments thereof) that bind a specific molecule that is a member of a binding pair may be used as diagnostics and prognostics, as described herein. In various embodiments, the present invention provides the measurement of a member of the binding pair, and the uses of such measurements in clinical applications. The immunoglobulins in the present invention may be used, for example, in the detection of an antigen in a biological sample whereby subjects may be tested for aberrant levels of the molecule to which the immunoglobulin binds. By “aberrant levels” is meant increased or decreased relative to that present, or a standard level representing that present, in an analogous sample from a portion of the body or from a subject not having the disease. The antibodies of this invention may also be included as a reagent in a kit for use in a diagnostic or prognostic technique.

In one embodiment, an antibody of the invention that immunospecifically binds to an infectious disease agent, such as Pneumocystis, or PCA1 protein or polypeptide may be used to diagnose, prognose or screen for the infectious disease.

Examples of suitable assays to detect the presence of the epitope include but are not limited to ELISA, radioimmunoassay, gel-diffusion precipitation reaction assay, immunodiffusion assay, agglutination assay, fluorescent immunoassay, protein A immunoassay, or immunoelectrophoresis assay.

The tissue or cell type to be analyzed will generally include those which are known, or suspected, to express the particular epitope. The protein isolation methods employed herein may, for example, be such as those described in Harlow and Lane, Antibodies: A Laboratory Manual, Cold Spring Harbor Laboratory Press, Cold Spring Harbor, N.Y. (1988), which is hereby incorporated by reference in its entirety. The isolated cells can be derived from cell culture or from a subject. The antibodies (or functionally active fragments thereof) useful in the present invention may, additionally, be employed histologically, as in immunofluorescence, immunohistochemistry, or immunoelectron microscopy, for in situ detection of the epitope or pathogens expressing the epitope. In situ detection may be accomplished by removing a histological specimen from a patient, such as paraffin embedded sections of affected tissues and applying thereto a labeled antibody of the present invention. The antibody (or functionally active fragment thereof) is preferably applied by overlaying the labeled antibody onto a biological sample. If the molecule to which the antibody binds is present in the cytoplasm, it may be desirable to introduce the antibody inside the cell, for example, by making the cell membrane permeable. Through the use of such a procedure, it is possible to determine not only the presence of the particular molecule, but also its distribution in the examined tissue. Using the present invention, those of ordinary skill will readily perceive that any of a wide variety of histological methods (such as staining procedures) can be modified to achieve such in situ detection of epitopes of PCA1 protein or polypeptide.

Immunoassays for the particular molecule will typically comprise incubating a sample, such as a biological fluid, a tissue extract, freshly harvested cells, or lysates of cultured cells, in the presence of a detectably labeled antibody and detecting the bound antibody by any of a number of techniques well-known in the art.

The biological sample may be brought in contact with and immobilized onto a solid phase support or carrier such as nitrocellulose, or other solid support which is capable of immobilizing cells, cell particles, or soluble proteins. The support may then be washed with suitable buffers followed by treatment with the detectably labeled antibody. The solid phase support may then be washed with the buffer a second time to remove unbound antibody. The amount of bound label on solid support may then be detected by conventional means. “Solid phase support or carrier” includes any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given antibody may be determined according to well known methods. Those skilled in the art will be able to determine operative and optimal assay conditions for each determination by employing routine experimentation.

One of the ways in which an antibody can be detectably labeled is by linking the same to an enzyme and use in an enzyme immunoassay (EIA) (Voller, “The Enzyme Linked Immunosorbent Assay (ELISA),” Diagnostic Horizons 2:1-7, Microbiological Associates Quarterly Publication, Walkersville, Md. (1978); Voller et al., J. Clin. Pathol. 31:507-520 (1978); Butler, Meth. Enzymol. 73:482-523 (1981); Maggio, E. (ed.), Enzyme Immunoassay, CRC Press, Boca Raton, Fla. (1980); Ishikawa et al., (eds.), Enzyme immunoassay, Kgaku Shoin, Tokyo (1981), each of which is hereby incorporated by reference in its entirety). The enzyme which is bound to the antibody will react with an appropriate substrate, preferably a chromogenic substrate, in such a manner as to produce a chemical moiety which can be detected, for example, by spectrophotometric, fluorimetric, or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or fragments, it is possible to detect the protein that the antibody was designed for through the use of a radioimmunoassay (RIA) (see, e.g., Weintraub, Principles of Radioimmunoassays, Seventh Training Course on Radioligand Assay Techniques, The Endocrine Society (1986), each of which is hereby incorporated by reference). The radioactive isotope can be detected by such means as the use of a gamma counter or a scintillation counter or by autoradiography. It is also possible to label the antibody with a fluorescent compound or semiconductor nanocrystals. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. A number of various semiconductor nanocrystals (i.e., nanodots) can be selected. Chemiluminescent compounds can alternatively be coupled to the antibodies. The presence of the chemiluminescent-tagged antibody is determined by detecting the presence of luminescence that arises during the course of a chemical reaction. Examples of particularly useful chemiluminescent labeling compounds are luminol, isoluminol, theromatic acridinium ester, imidazole, acridinium salt, and oxalate ester.

Likewise, a bioluminescent compound may be used to label the synthetic antibody of the present invention. Bioluminescence is a type of chemiluminescence found in biological systems, in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

A further aspect of the present invention relates to an isolated protein or polypeptide comprising the amino acid sequence of SEQ ID NO:1.

In one embodiment, the isolated protein or polypeptide is PCA1 from a source other than mouse (murine) and while it may have an amino acid sequence that is quite varied from SEQ ID NO:1, it has a conserved cysteine backbone, as described supra.

PCA1 protein of the present invention may be isolated from a cell or expressed. Expression of a PCA1 protein or polypeptide of the present invention can be carried out by introducing a nucleic acid molecule encoding the PCA1 protein or polypeptide into an expression system of choice using conventional recombinant technology. Generally, this involves inserting the nucleic acid molecule into an expression system to which the molecule is heterologous (i.e., not normally present).

The nucleic acid sequence (GenBank Accession No. KX011348.1, which is hereby incorporated by reference in its entirety) encoding SEQ ID NO:1, identified herein as SEQ ID NO:2, is as follows:

atgtttttct taagaatcat ctttatattt
atttttttaa aaatatcata tgcagaaaac
acagataaac tctcagattt cgaaaaaaaa
tatccagaat tatatcaagc aaatccacat
gctttaaaac tggaagcatt gaaaagcgga
ttttcaggca aatctgtaaa aaaaggattg
ggtgtttttc atatagggaa tcttggtcat
tatagagatc ataaaccagt tatattgcat
gtaattatgg gattaactgt tggactcgca
gagtgtcgcg ggacactcgc cgaaagatgt
aaagtcataa aagccctagg aaatccaata
acacaatatt gcaataaacc atatgataca
tgccaagatt attttgacgc tcgaaattac
ttactcccta tgaaagatca attaaaaaac
ccacacgccc atcatgatgc atgcagaacg
attttgctaa attgcctctt ttttaaacat
cgtaattata ttacttccga ttgtgttcct
ttggtagcat tatgttattt gcgggttcgt
caaaactttg tagaagcaat tatgaccgaa
gcattaagag gggaaattaa tactaagggt
gctgctgcag caatgaaaaa agtatgtgaa
aaaattggac atgagagtcc ggacttgctt
catttatgtt ttaagaccac tgtattagaa
aaacctaaaa ggtctaataa acagtatatt
gaagatgtta agtcaagaat aaggacagtt
tcgactggaa attgccgtca ggttttggaa
gaatgctatt ttaatgttct agattatcca
gatatttatc aatcatgtag gaattttcga
cgattctgtt cagaaatagg agttgtatat
actccagtcg attccacttt tgatttattt
cagaagcccc tttctgcaga aaagttacta
attgatactt cttcaaaaat ctcagaagac
ttaggtcttg gtttttctaa atatgtacaa
aaaaaatcaa gcaatcttga gattgcggca
tatttagtta ataagacttg ggtctatgat
aatgattgca gaaataaatt aaaagaacta
tgtctgcata ttgcttctct accgcttaca
aaacaactat gcacattagc acatgataga
aattcgaaac tctgtaggga tttttataac
tctattggga ctgaatgcta ttctttatat
tatgaattta agaatgttgg attattatac
aattatactt atcgtctttc aagagatcaa
tgctctaaat atgtagaaag atgtcttttt
cttagggagc aatatgctta ttggaattct
ctagatactt gtgctaatgt attttcttca
tgttataaag aagatatgga tttttcagcc
aaattagatc ttctaaatag gataaaagat
aagattgtag ttccaaaagg aaacacgagg
tattttgtag agttattgtg taaaagctat
attgtcgccg aatgcagcgc cagtgattta
atgttcaaat cttatgctct tatggaagcc
tgtcttcacc cagaaaggat ctgtagagaa
ttaaaaaatc atttttccga agaatctagg
aaattagaaa ataaattaag gagtatttta
aaacccacat attatgaatg caaagatcta
ggacaaaagt gcaactctgg attttatttt
gatggagata tagaagctca atgcaatcat
ttcaaaaaaa gatgtcaaga taaacaagag
agactaaaat taattaatca tattgttgat
tcatctgctc tttatctcgc aaatgaagta
caatgcagaa cttatttcga cagtttttgt
ggtgcgaatg taaaacaaga attcaaacaa
atatgcaaca aaggagctaa tggcatatgc
cctgatataa tagatgattc taaagaacat
tgtgctcatt tgattaatca tttaacatct
cttggaattt catcgtcttc tgcttcactt
ccattggact attgcgactc agcgattaat
tactgtaatt ctctttcgaa gttttgcacg
gaatcaaaac gacagtgcga ttctgttatt
tctttctgca ctagcgaatc aaaaaaaact
gatgaatatg gttcttttat tgaccaatat
cccgcggctg cagcaaatgc aaccaaatgc
aaggtaactt tgaaagagtt atgccaagat
tcaagcaaaa aagactctta ttcaacacta
tgtgcttata ataaagatgg ttataccgaa
atatgtaaaa acttaagaaa tttcatagaa
aaagcatgcg agaatttgag aattcattta
catacttatg atacaaactc actcaatacg
aataaaggat ctgctcaaga tagatgcact
tatataagaa atctttactt taaatttaaa
aatatatgtt tattggttga tcctttctat
gacttatctc ctattatcac tcaagaatgt
aaaaccaata tatccgaacc agcactgcct
gataaggatc ctcaacctac atcttcacct
cagccaaaac ctcggccaag acctcgacct
caacctcaac ctcatccaca tccaaaacct
cagcctcagc cgacgccaga acctcagcct
cagccggcgc cagaacctcg acctcagccg
acgtcaaaac ctcgacctca gccaacgtca
aaacctcgac ctcagccgac gccagaacct
cgacctctgc cggtgccagg acctggacct
ctgccggtgc caggacctcg acctcaacct
caacctcaac ctcaacctca gcctcaacct
caacctcagc ctcaacctca acctcagcct
cagcctcagc ctcagcctca gcctcaacct
cagccgaagc ctcaaccacc atctcagtca
acatcagaat cagcatcgca atccaaacca
aaaccaacaa cacaaacaaa accgtcaccg
agaccacacc caaagccggt gccaaaacca
tcatcgatag acacaggacc atcaaaatcg
gattcaagct tcatttttac agtaacaaaa
acaataacaa agatatcaga aacagaaaaa
ccatctacaa aaccatctgt gaaaccaacc
tctacaaaga caacatcaaa accatctaca
aaaccatcta caaaaccatc tgtaaaacca
gcctctacaa agacaacatc agaatcagaa
aaaccaacat tggaagaagt tccagaaact
aaagggaatg gtgtaagagt aataggattt
gaggggttac aattattatc aatgattgtt
gcaataataa ttgggatatg gataatgtaa

The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted PCA1 protein or polypeptide coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/−(see “Stratagene Cloning Systems” Catalog (1993) from Stratagene, La Jolla, Calif., which is hereby incorporated by reference in its entirety), pQE, pIH821, pGEX, pFastBac series (Invitrogen), pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the PCA1 protein or polypeptide-encoding sequence in a cell. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is desirable to use strong promoters to obtain a high level of transcription and, hence, expression of the gene. Depending upon the host cell system utilized, any one of a number of suitable promoters may be used. For instance, when cloning in E. coli, its bacteriophages, or plasmids, promoters such as the PH promoter, T7 phage promoter, lac promoter, trp promoter, rec A promoter, ribosomal RNA promoter, the P_{R} and P_{L} promoters of coliphage lambda and others including, but not limited to, lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

The PCA1 protein or polypeptide-encoding nucleic acid, a promoter molecule of choice, a suitable 3′ regulatory region and, if desired, a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a PCA1 protein or polypeptide of the present invention is inserted into a vector in the sense (i.e., 5′→3′) direction, such that the open reading frame is properly oriented for the expression of the encoded PCA1 protein or polypeptide of the present invention under the control of a promoter of choice. Single or multiple nucleic acids may be ligated into an appropriate vector in this way, under the control of a suitable promoter, to prepare a nucleic acid construct.

Once the isolated nucleic acid molecule encoding the PCA1 protein or polypeptide of the present invention has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The DNA sequences are incorporated into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

A further aspect of the present invention relates to a pharmaceutical composition comprising an isolated PCA1 protein or polypeptide of the present invention and a pharmaceutically acceptable carrier.

In one embodiment, the pharmaceutical composition includes, but is not limited to, pharmaceutically suitable adjuvants, carriers, excipients, or stabilizers (collectively referred hereinafter as “carrier”). The pharmaceutical compositions are preferably, though not necessarily, in liquid form such as solutions, suspensions, or emulsions. Typically, the composition will contain from about 0.01 to 99 percent, preferably from about 20 to 75 percent of one or more of the above-listed active agents, together with the adjuvants, carriers, excipients, stabilizers, etc.

The pharmaceutical compositions of the present invention can take any of a variety of known forms that are suitable for a particular mode of administration. Exemplary modes of administration include, without limitation, orally, by inhalation, by intranasal instillation, topically, transdermally, parenterally, subcutaneously, intravenous injection, intra-arterial injection, intramuscular injection, intraplurally, intraperitoneally, by intracavitary or intravesical instillation, intraocularly, intraventricularly, intralesionally, intraspinally, or by application to mucous membranes, such as, that of the nose, throat, and bronchial tubes.

The pharmaceutical forms suitable for injectable use (e.g., intravenous, intra-arterial, intramuscular, etc.) include sterile aqueous solutions or dispersions and sterile powders for the extemporaneous preparation of sterile injectable solutions or dispersions. The form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to, sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

Oral dosage formulations can include standard carriers such as pharmaceutical grades of mannitol, lactose, starch, magnesium stearate, sodium saccharine, cellulose, magnesium carbonate, etc. Suitable carriers include lubricants and inert fillers such as lactose, sucrose, or cornstarch. In another embodiment, these compounds are tableted with conventional tablet bases such as lactose, sucrose, or cornstarch in combination with binders like acacia, gum gragacanth, cornstarch, or gelatin; disintegrating agents such as cornstarch, potato starch, or alginic acid; a lubricant like stearic acid or magnesium stearate; and sweetening agents such as sucrose, lactose, or saccharine; and flavoring agents such as peppermint oil, oil of wintergreen, or artificial flavorings. Generally, the ingredients are supplied either separately or mixed together in unit dosage form, for example, as a dry lyophilized powder or water free concentrate in a hermetically sealed container such as an ampule or sachette indicating the quantity of active agent.

For use as aerosols, the active agents in solution or suspension may be packaged in a pressurized aerosol container together with suitable propellants, for example, hydrocarbon propellants like propane, butane, or isobutane with conventional adjuvants. The active agents of the present invention also may be administered in a non-pressurized form such as in a nebulizer or atomizer.

For parenteral administration, aqueous solutions in water-soluble form can be used to deliver one or more of the active agents. Additionally, suspensions of the active agent(s) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

In addition to the formulations described previously, the active agent(s) may also be formulated as a depot preparation. Such long acting formulations may be administered by implantation (e.g., subcutaneously or intramuscularly) or by intramuscular injection. Thus, for example, the active agent(s) may be formulated with suitable polymeric or hydrophobic materials (e.g., as an emulsion in an acceptable oil) or ion exchange resins, or as sparingly soluble derivatives (e.g., as a sparingly soluble salt). Selection of polymeric matrix material is based on biocompatibility, biodegradability, mechanical properties, cosmetic appearance, and interface properties. The particular application of the active agent(s) will define the appropriate formulation. Potential matrices for the compositions may be biodegradable and chemically defined calcium sulfate, tricalcium phosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate, as well as other materials that are known in the drug delivery arts. The bioceramics may be altered in composition, such as in calcium-aluminate-phosphate and processing to alter pore size, particle size, particle shape, and biodegradability.

The above-identified active agents are to be administered in an amount effective to achieve their intended purpose (i.e., to treat infection, treat a subject at risk for infection, induce an active immune response, or provide passive immunity). While individual needs vary, determination of optimal ranges of effective amounts of each component is within the skill of the art. The quantity administered will vary depending on the patient and the mode of administration and can be any effective amount. In one embodiment, a typical dosage may include about 0.1 to about 100 mg/kg·body wt. In another embodiment, the preferred dosage may include about 1 to about 50 mg/kg·body wt. However, because patients respond differently to therapies, monitoring of the treatment efficacy should be conducted, allowing for adjustment of the dosages as needed. Treatment regimen for the administration of the above-identified active agents of the present invention can also be determined readily by those with ordinary skill in art.

### EXAMPLES

The following examples are provided to illustrate embodiments of the present invention, but they are by no means intended to limit its scope.

### Example 1—Immunization with Peal Protects Mice Against Pneumocystis Pneumonia and Generates Antibody to Pneumocystis Jirovecii

Materials and Methods

Mice.

Pathogen-free CB.17 and SCID mice on a CB.17 background were obtained from a breeding colony at the University of Rochester animal care facilities (originally purchased from Taconic Biosciences, Germantown, N.Y.) and housed in microisolator cages, given sterile food and water ad lib.

Cloning and Characterization of the Pca1 Antigen cDNA and Gene.

Using the Pca1 partial cDNA sequence (GenBank Accession No. AY371664.1, which is hereby incorporated by reference in its entirety) as a starting point, GeneRacer™ (Invitrogen, Carlsbad, Calif.) reactions were used to obtain the full-length sequence of the cDNA (GenBank Accession No. KX011348, which is hereby incorporated by reference in its entirety). Based on the cDNA sequence, primers were designed to amplify the full-length Pca1 gene. All procedures for cDNA synthesis and genomic DNA isolation have been described previously (Wells et al., “Active Immunization Against Pneumocystis Carinii With a Recombinant P. Carinii Antigen,” Infect. Immun. 74:2446-8 (2006); Wells et al., “Epitope Mapping of a Protective Monoclonal Antibody Against Pneumocystis Carinii With Shared Reactivity to Streptococcus Pneumoniae Surface Antigen PspA,” Infect. Immun. 72:1548-56 (2004), which are hereby incorporated by reference in their entirety). Southern blotting was used to determine the copy number of the Pca1 gene in the P. murina genome as described previously (Lee et al., “Molecular Characterization of KEX1, a Kexin-Like Protease in Mouse Pneumocystis Carinii,” Gene 242:141-50 (2000), which is hereby incorporated by reference in its entirety).

Cloning and Expression in E. coli.

Expressing the recombinant full-length Pca1 protein proved difficult. For the immunization model, a 544-aa N-terminal portion of the Pca1 protein (19-1650 bp) was produced by GenScript (Piscataway, N.J.) as a fusion protein with Trigger Factor (TF) using proprietary technology to produce a codon-optimized synthetic gene based on the Pca1 amino acid sequence. TF was also expressed and purified using the same system to serve as a control. To express a portion of the Pca1 gene without the fusion partner, DNA was generated from the codon-optimized synthetic gene by PCR using a 44-bp primer pair which introduced unique Xhol restriction sites. The resulting PCR product was subcloned into the pET14b (Novagen, Gibbstown, N.J.) expression vector forming a sequence encoding a 388-aa portion (274-1439 bp) of Pca1 with an N-terminal His₆-tag. It was then transformed in the BL21 (DE3) RIL Codon Plus (New England BioLabs, Ipswich, Mass.) host to allow for optimal expression of AT-rich genes.

Immunization and Infectious Challenge Model.

To investigate the immunogenicity and efficacy of the recombinant Pca1 fusion protein, groups of 5-10 six to eight week old female CB.17 mice were immunized subcutaneously with three doses of 100 μg of Pca1 emulsified in TiterMax™ Gold adjuvant (Sigma, St. Louis, Mo.) given in three week intervals. To evaluate the dose-response, additional mice were immunized with 10, 40 or 80 μg of Pca1. Control mice were immunized with the fusion partner alone, an unrelated protein or whole Pc. Sera was obtained from the mice by submandibular venipuncture three weeks after the final dose and stored at −80° C. until use. Two weeks after the final immunization, all mice were CD4⁺ T cell-depleted through twice weekly injections of 250 μg anti-CD4 monoclonal antibody (clone GK1.5; ATCC, Manassas, Va.). CD4⁺ T cell depletion was evaluated by flow cytometry analysis of lymphocytes present in the spleen and bronchoalveolar lavage fluid. The mice were subsequently exposed to Pc while being co-housed with Pc-infected SCID mice for two weeks. This simulates the natural route of Pc infection. The mice were monitored for signs of infection including weight loss and increased respiratory rate while CD4⁺ T cell depletion was maintained. The mice were sacrificed after demonstrating signs of active infection, four to six weeks after Pc exposure. At the time of sacrifice, serum was collected via cardiac puncture, spleens were removed, bronchoalveolar lavage was performed, and lungs were removed and flash frozen for Pc burden enumeration.

Serology.

Sera from immunized mice was assayed by enzyme-linked immunosorbent assay (ELISA) as previously described (Harmsen et al., “Active Immunity to Pneumocystis Carinii Reinfection in T-Cell-Depleted Mice,” Infect. Immun. 63:2391-5 (1995), which is hereby incorporated by reference in its entirety) for Pca1-specific antibody production by coating 96-well plates with 1 μg/mL of the 388-aa length portion of the Pca1 protein not containing the fusion partner. An immunofluorescence assay was used to determine if immunization induced antibody to P. murina, P. carinii and P. jirovecii as previously described (Gigliotti et al., “Passive Intranasal Monoclonal Antibody Prophylaxis Against Murine Pneumocystis Carinii Pneumonia,” Infect. Immun. 70:1069-74 (2002), which is hereby incorporated by reference in its entirety).

Quantitation of P. murina.

Homogenized lung tissue was prepared and P. murina burden was calculated using quantitative real-time PCR of the single copy kex1 gene as previously described (Gigliotti et al., “Characterization of Transmission of Pneumocystis carinii f sp. Muris Through Immunocompetent BALB/c Mice,” Infect. Immun. 71:3852-6 (2003), which is hereby incorporated by reference in its entirety). The limit of detection for this assay has been determined to be 4 log₁₀ copies of kex1 gene per total lung volume.

Statistical Analysis.

Categorical data were analyzed by Fisher's exact test. Continuous data were analyzed by t test. P-values <0.05 were considered significant. Analysis was performed with GraphPad Prism v. 6 software (La Jolla, Calif.).

Study Approval.

All procedures performed were subject to University of Rochester Committee on Animal Resources approval.

Accession Number.

The genomic DNA sequence of Pca1 has been deposited in GenBank under accession number KX011348, which is hereby incorporated by reference in its entirety.

Results

Pca1 Immunization Provides Protection Against Infection.

After immunizing immunocompetent mice with either Pca1, whole Pc or control proteins, the human susceptibility to PcP was mimicked through depletion of CD4⁺ T cells with anti-CD4 antibody (clone GK1.5). Circulating CD4⁺ T cells were reduced from 13-18% to 0-1% of the total lymphocyte population sampled from the spleen. An established mouse model of PcP was then used that simulates natural infection by co-housing the susceptible, immunized mice with actively infected SCID mice (Wells et al., “Active Immunization Against Pneumocystis Carinii With a Recombinant P. Carinii Antigen,” Infect Immun 74:2446-8 (2006), which is hereby incorporated by reference in its entirety). Pca1 immunization provided protection against subsequent infectious challenge (Table 1), with complete protection being defined as undetectable organism burden by qPCR (total lung organism burden below 10⁴, the lower limit of detection). All unimmunized control mice developed PcP, confirming the functional efficacy of the CD4⁺ cell depletion regimen and infection strategy. Nearly all mice (32/37) immunized with Pca1 were completely protected against infection with undetectable organism burden at the time of sacrifice, whereas none of the 28 control protein-immunized mice were protected (Table 1, p<0.0001, Fisher's exact test). The proportion of mice protected by Pca1 immunization was statistically indistinguishable from the proportion protected by whole cell Pc immunization (Table 1). These results were confirmed by examining the lung homogenates after silver staining to identify Pc cysts.

TABLE 1
Summary of Protection by Pca1 Immunization - Defined as
Organism Burden Below the Limit of Detection by qPCR

| | Immunogen | | |
|---|---|---|---|
| Proportion of mice | Negative | Pca1 Fusion | Whole Pc |
| protected | Control Protein | Protein | Positive Control |
| No. not protected | 23 | 4 | 1 |
| No. protected | 0 | 33 | 11 |
| Total No. mice | 23 | 37 | 12 |
| Percent Protected | 0% | 89%*** | 92%*** |

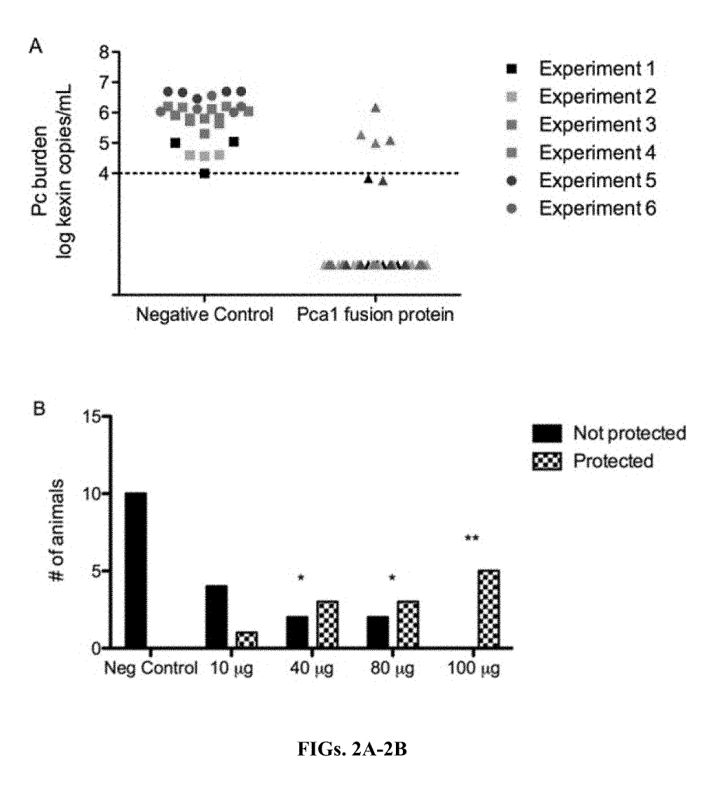

Furthermore, PCR for the multicopy gpA gene failed to detect any target DNA in lung samples from protected mice. The finding of reduced organism burden compared to experimental controls in the few mice immunized with Pca1 but not completely protected may be an in vivo demonstration of dose-dependent response to immunization (FIG. 2A). Although not statistically significant likely due to sample size, this provides additional evidence for vaccine efficacy.

To determine a threshold dose, mice were immunized with varying doses of Pca1. As expected, the efficacy of Pca1 was dose-dependent. All five mice immunized with 100 μg of Pca1 were completely protected against infection, whereas only three of five mice immunized with either 80 μg or 40 μg and one of five mice immunized with 10 μg of Pca1 had undetectable burden (FIG. 2B).

Molecular Characterization of P. murina Pca1.

Nucleotide sequence analysis (GenBak Accession No. KX011348, which is hereby incorporated by reference in its entirety) and Southern blotting demonstrated that the Pca1 antigen gene is present in a single copy. It encodes a 1099-amino acid protein of unknown function. Specifically, it does not have characteristics typical of fungal adhesins (Ramana and Gupta, “FaaPred: A SVM-Based Prediction Method for Fungal Adhesins and Adhesin-Like Proteins,” PLoS One 5:e9695 (2010), which is hereby incorporated by reference in its entirety). Nor was there significant homology with closely related fungal species such as S. pombe. Significant protein sequence homology was found in the genomes of rat Pc (P. carinii) and human Pc (P. jirovecii) deposited sequences. A single protein with identity and similarity to Pca1 of 39% and 61%, respectively, was identified in the P. carinii genome (KTW27087.1). Five putative Pca1 orthologs were identified in the genome of P. jirovecii (KTW31106.1, KTW25468.1, KTW32226.1, KTW25482.1, KTW25472.1, which are hereby incorporated by reference in their entirety). These proteins displayed identity and similarity to Pca1 of up to 26% and 45%, respectively, and 48 of the 49 cysteine residues in mouse Pc Pca1 are conserved in these human Pc sequences. Importantly, these five P. jirovecii proteins were highly similar to each other, with identity and similarity scores ranging from 78-89% and 87-93%, respectively.

Pca1 Immunization Generates Antigen-Specific Antibody that Cross-Reacts with the Human Pathogen, P. jirovecii.

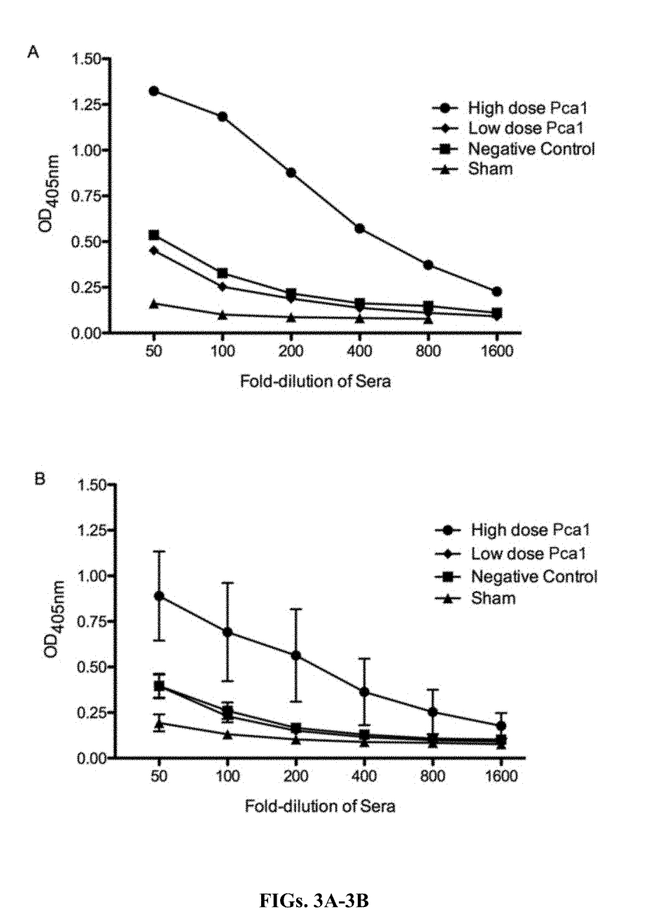

Pca1 immunization resulted in antibody production to the Pca1 portion of the fusion protein (FIG. 3A) in a dose-dependent fashion after a three dose immunization series as measured by ELISA to a Pca1 polypeptide lacking the fusion partner. Little Pca1-specific antibody could be detected in sera from mice immunized with a low dose (10 μg) of Pca1 or the fusion partner alone. Pca1-specific antibody remained detectable in high dose Pca1-immunized mice following Pc exposure and sacrifice approximately nine weeks after the last immunization (FIG. 3B).

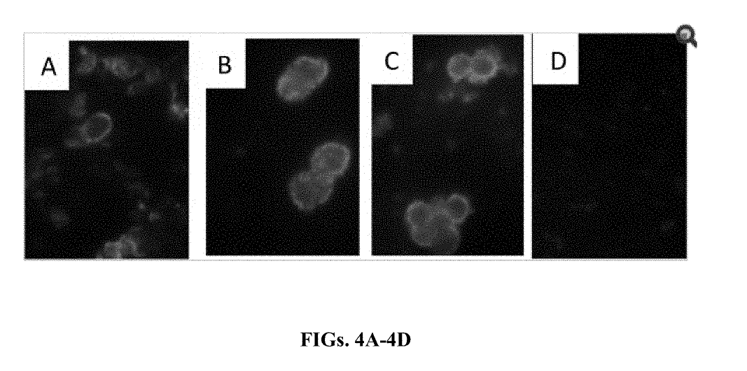

To evaluate cross-reactivity, indirect immunofluorescence assays (IFA) were performed with antisera from Pca1- and control protein-immunized mice and tested against Pc isolated from three different host species. Antisera from Pca1-immunized mice contained antibody that bound to not only the mouse-derived P. murina (FIG. 4A) cysts but, also rat-derived P. carinii (FIG. 4B) and, importantly, to four distinct clinical isolates of P. jirovecii isolated from infected patients (FIG. 4C). This binding to P. jirovecii was similar to that seen with positive control, known cross-reactive Mab 4F11. No such binding to P. jirovecii was observed with the antisera from mice immunized with the fusion partner, trigger factor (FIG. 4D).

### DISCUSSION

As described herein, the characteristics of a novel Pc antigen, Pca1, isolated from P. murina with an ortholog identified in P. jiroveci has been described. Immunization with the N-terminal half of Pca1 protects in a mouse model of PcP and has the unique characteristic of inducing antibody that cross reacts with human Pc, P. jirovecii. The host species specificity of Pc impedes the human translation of many animal model observations, and previous studies have failed to demonstrate serologic cross reactivity or protection (Gigliotti and Harmsen, “Pneumocystis Carinii Host Origin Defines the Antibody Specificity and Protective Response Induced by Immunization,” J. Infect. Dis. 176:1322-6 (1997), which is hereby incorporated by reference in its entirety). Only three cross-reactive Mabs have been identified to date (Gigliotti and Harmsen, “Pneumocystis Carinii Host Origin Defines the Antibody Specificity and Protective Response Induced by Immunization,” J. Infect. Dis. 176:1322-6 (1997); Gigliotti et al., “Development of Murine Monoclonal Antibodies to Pneumocystis Carinii,” J. Infect. Dis. 154:315-22 (1986), which are hereby incorporated by reference in their entirety). One of these antibodies, 4F11, has a known antibody epitope in the C-terminus of the full length Pca1. Since immunization with this polypeptide was only partially protective in the mouse model (Wells et al., “Epitope Mapping of a Protective Monoclonal Antibody Against Pneumocystis Carinii With Shared Reactivity to Streptococcus Pneumoniae Surface Antigen PspA,” Infect. Immun. 72:1548-56 (2004), which is hereby incorporated by reference in its entirety), the present study set out to clone the entire gene. The cross reactivity of N-terminal Pca1 antisera opens the possibility of using heterologous Pc antigens to protect humans against the development of PcP.

The extent of protection that resulted from immunization with Pca1 was unexpected since partial protection after immunization appears to be the typical outcome of immunization against fungal pathogens such as Candida, Aspergillus, and Cryptococcus (Rachini et al., “An Anti-Beta-Glucan Monoclonal Antibody Inhibits Growth and Capsule Formation of Cryptococcus Neoformans In Vitro and Exerts Therapeutic, Anticryptococcal Activity In Vivo,” Infect. Immun. 75:5085-94 (2007); Torosantucci et al., “A Novel Glyco-Conjugate Vaccine Against Fungal Pathogens,” J. Exp. Med. 202:597-606 (2005); Xin and Cutler, “Vaccine and Monoclonal Antibody That Enhance Mouse Resistance to Candidiasis,” Clin. Vaccine Immunol. 18:1656-67 (2011); Spellberg et al., “Efficacy of the Anti-Candida rA1s3p-N or rA1s1p-N Vaccines Against Disseminated and Mucosal Candidiasis,” J. Infect. Dis. 194:256-60 (2006); Cassone and Casadevall, “Recent Progress in Vaccines Against Fungal Diseases,” Curr. Opin. Microbiol. 15:427-33 (2012), which are hereby incorporated by reference in their entirety). A number of potential vaccine candidates have been evaluated in animal models of PcP as well, but at best achieved only partial protection (Wells et al., “Active Immunization Against Pneumocystis Carinii With a Recombinant P. Carinii Antigen,” Infect Immun 74:2446-8 (2006); Duan et al., “Protective Effect of DNA Vaccine With the Gene Encoding 55 kDa Antigen Fragment Against Pneumocystis Carinii in Mice,” Asian Pac. J. Trop. Med. 4:353-6 (2011); Feng et al., “Active Immunization Against Pneumocystis Carinii With p55-v3 DNA Vaccine in Rats,” Can. J. Microbiol. 57:375-81 (2011); Gigliotti et al., “Immunization with Pneumocystis Carinii gpA is Immunogenic but not Protective in a Mouse Model of P. Carinii Pneumonia,” Infect. Immun. 66:3179-82 (1998); Smulian et al., “Immunization With Recombinant Pneumocystis Carinii p55 Antigen Provides Partial Protection Against Infection: Characterization of Epitope Recognition Associated With Immunization,” Microbes Infect. 2:127-36 (2000); Zheng et al., “CD4+ T Cell-Independent DNA Vaccination Against Opportunistic Infections,” J. Clin. Invest. 115:3536-44 (2005), which are hereby incorporated by reference in their entirety). A recent publication also describes boosting naturally acquired antibody in Pc-exposed animals with a fragment of kexin as a means to enhance immune response to Pc (Kling and Norris, “Vaccine-Induced Immunogenicity and Protection Against Pneumocystis Pneumonia in a Nonhuman Primate Model of HIV and Pneumocystis Coinfection,” J. Infect. Dis. 213(10):1586-95 (2016), which is hereby incorporated by reference in its entirety). The presence of shared antibody epitopes between kexin and Pca1 warrants further analysis of vaccine immunogenicity and efficacy (Wells et al., “Epitope Mapping of a Protective Monoclonal Antibody Against Pneumocystis Carinii With Shared Reactivity to Streptococcus Pneumoniae Surface Antigen PspA,” Infect. Immun. 72:1548-56 (2004), which is hereby incorporated by reference in ints entirety). The complete clearance of Pc from Pca1-immunized and exposed animals reinforces the role Pca1 may have in PcP protection.

While these experiments were not designed to determine the mechanism of vaccine-induced protection, several observations are consistent with antibody-mediated protection. It has previously been shown that administration of a Pca1-binding Mab (4F11) reduces organism burden, and maximal reduction of Pc after Mab infusion requires an intact Fc region and a functional complement system (Wells et al., “Complement and Fc Function are Required for Optimal Antibody Prophylaxis Against Pneumocystis Carinii Pneumonia,” Infect. Immun. 74:390-3 (2006), which is hereby incorporated by reference in its entirety). Persistence of protection well after immunization and in the absence of CD4⁺ T cells also is most consistent with antibody-mediated protection. CD8⁺ T cells do not appear to have a major role in defense against Pc and are not necessary for organism clearance. Therefore they are unlikely to play a role in Pca1-mediated protection (Gigliotti et al., “Sensitized CD8+ T Cells Fail to Control Organism Burden but Accelerate the Onset of Lung Injury During Pneumocystis Carinii Pneumonia,” Infect. Immun. 74:6310-6 (2006), which is hereby incorporated by reference in its entirety).

To circumvent the difficulties in synthesizing full length Pc proteins, the immunogenicity of fragments of Pca1 have been examined. It was previously demonstrated that the C-terminal end was partially protective (Wells et al., “Active Immunization Against Pneumocystis Carinii With a Recombinant P. Carinii Antigen,” Infect Immun 74:2446-8 (2006), which is hereby incorporated by reference in its entirety), and the efficacy of immunization with the N-terminal half of the protein has now been characterized. In addition to completely protecting nearly all animals from subsequent infection, Pca1 immunization also produced antibodies which reacted with P. jirovecii, a rare finding. The ability of Pca1 immunization to protect mice from infection and induce antibody that binds to P. jirovecii makes Pca1 a lead candidate for further development into a potential human subunit vaccine.

Furthermore, the ortholog of mouse Pca1 in the human pathogen, P. jiroveci, appears to have been identified. The degree of identity and similarity observed between mouse Pc-derived Pca1 and candidate ortholog sequences identified in rat and human Pc is similar to that observed between Pc surface glycoprotein A (gpA) sequences from different mammalian host species (Wright et al., “Conserved Sequence Homology of Cysteine-Rich Regions in Genes Encoding Glycoprotein A in Pneumocystis Carinii Derived From Different Host Species,” Infect. Immun. 62:1513-9 (1994); Wright et al., “Cloning and Characterization of a Conserved Region of Human and Rhesus Macaque Pneumocystis Carinii gpA,” Gene 167:185-9 (1995), which is hereby incorporated by reference in its entirety). The additional observation of cysteine conservation between mouse Pca1 and the candidate human Pc ortholog suggests similar protein structure. The identification of a human Pc ortholog and demonstration of cross-reactive antibody production overcomes the host species specificity impediment to translation of Pc animal models.

It has been demonstrated that active immunization with mouse Pca1 protects mice against subsequent infectious challenge. The mice were immunocompetent at the time of immunization. However, most patients at risk for PcP can be identified during periods of relative immunocompetency, such as during early HIV infection or prior to the administration of immunosuppressive agents for malignancy or autoimmune conditions. These patients would be ideal candidates for active immunization. Even patients undergoing chemotherapy have been shown to demonstrate adequate antibody responses to polysaccharide, protein and conjugated vaccines (Feldman et al., “Risk of Haemophilus Influenzae Type B Disease in Children With Cancer and Response of Immunocompromised Leukemic Children to a Conjugate Vaccine,” J. Infect. Dis. 161:926-31 (1990); Nordoy et al., “Cancer Patients Undergoing Chemotherapy Show Adequate Serological Response to Vaccinations Against Influenza Virus and Streptococcus Pneumoniae,” Med. Oncol. 19:71-8 (2002); Berglund et al., “The Response to Vaccination Against Influenza A (H1N1) 2009, Seasonal Influenza and Streptococcus Pneumoniae in Adult Outpatients With Ongoing Treatment for Cancer With and Without Rituximab,”Acta. Oncol. 53:1212-20 (2014), which are hereby incorporated by reference in their entirety). The vaccination strategy could also be optimized to function in a T-independent fashion through adjuvants of conjugation. Additionally, it could be used in CD4⁺ T cell-independent platforms such as DNA-vaccination (Zheng et al., “CD4+ T Cell-Independent DNA Vaccination Against Opportunistic Infections,” J. Clin. Invest. 115:3536-44 (2005), which is hereby incorporated by reference in its entirety).

Immune-mediated inflammation is a key component in the pathophysiology of PcP. Passive treatment with pools of anti-Pc monoclonal antibodies including 4F11 has previously been shown to reduce both organism burden as well as inflammation (Gigliotti et al., “Passive Intranasal Monoclonal Antibody Prophylaxis Against Murine Pneumocystis Carinii Pneumonia,” Infect. Immun. 70:1069-74 (2002); Gigliotti and Hughes, “Passive Immunoprophylaxis With Specific Monoclonal Antibody Confers Partial Protection Against Pneumocystis Carinii Pneumonitis in Animal Models,” J. Clin. Invest. 81:1666-8 (1988); Empey et al., “Passive Immunization of Neonatal Mice Against Pneumocystis Carinii f sp. Muris Enhances Control of Infection Without Stimulating Inflammation,” Infect. Immun. 72:6211-20 (2004), which are hereby incorporated by reference in their entirety). Therefore, in addition to the antimicrobial effects, the immunoregulatory effects of high dose IVIG may provide additional benefit in the treatment of a patient with PcP. Pca1 or the human ortholog could be used to generate high titer antisera for passive administration as is done for diseases such as tetanus and rabies. Patients likely to have a poor prognosis, usually as a result of inflammatory injury, can frequently be identified on admission to the hospital and could be targeted for treatment (Armstrong-James et al., “A Prognostic Scoring Tool for Identification of Patients at High and Low Risk of Death From HIV-Associated Pneumocystis Jirovecii Pneumonia,” Int. J STD. AIDS 22:628-34 (2011), which is hereby incorporated by reference in its entirety). These studies support further investigation into the development of Pca1-based prophylactic and therapeutic immunotherapies. With PcP-related mortality remaining high and relatively unchanged, Pca1-based immunotherapy could provide a novel therapeutic approach to our current management of PcP.

### Example 2—Combined Treatment with Passive Antibody and the Anti-Inflammatory Agent Sulfasalazine Modulates Pulmonary Inflammation and Accelerates the Resolution of Pneumocystis Pneumonia

Materials and Methods

Mouse Model of Pneumocystis Pneumonia.

Severe combined immune deficient (SCID) mice on a C.B-17 background are maintained in a specific pathogen-free colony at University of Rochester Medical Center. For these studies, 6-8 week old female mice were infected with Pneumocystis by intratracheal inoculation with 1×10⁵ Pneumocystis cysts and then also cohoused with other Pneumocystis-infected SCID mice to ensure infection. The mice were fed acidified water and sterile food. Immune reconstitution (IR) was initiated three weeks after infection by giving mice an intraperitoneal (i.p.) injection of 5×10⁷ splenocytes from wild-type C.B-17 mice. All animals involved were treated ethically according to the guidelines from the Association for Assessment and Accreditation of Laboratory Care International (AAALAC International), and the protocols used were approved by the University Committee on Animal Resources.

Specific Anti Pneumocystis Antibody and Sulfasalazine Administration.

Specific anti-Pneumocystis antibodies have been developed and characterized (Table 2).

TABLE 2
Specific Anti-Pc Monoclonal Antibody Pool

| Antibody | Isotype | Epitope | Reference |
|---|---|---|---|
| 4F11(G1) | IgG1 | A12 | Wells et al., “Epitope Mapping of a Protective |
| | | | Monoclonal Antibody Against Pneumocystis |
| | | | Carinii With Shared Reactivity to Streptococcus |
| | | | Pneumoniae Surface Antigen PspA,” Infect. |
| | | | Immun. 72: 1548-56 (2004), which is hereby |
| | | | incorporated by reference in its entirety |
| 1G12 | IgG2b | 40-60 kDa | Gigliotti et al., “Development of Murine |
| | | | Monoclonal Antibodies to Pneumocystis Carinii,” |
| | | | J. Infect. Dis. 154: 315-22 (1986), which is hereby |
| | | | incorporated by reference in its entirety |
| 1F2 | IgG2a | 40-60 kDa | None |
| 1F5 | IgG1 | 40-60 kDa | None |
| 2B5 | IgG1 | gpA | Gigliotti et al., “Development of Murine |
| | | | Monoclonal Antibodies to Pneumocystis Carinii,” |
| | | | J. Infect. Dis. 154: 315-22 (1986), which is hereby |
| | | | incorporated by reference in its entirety |
| 3D6 | IgG1 | gpA | None |
| 1G4 | IgM | A12 | Gigliotti et al., “Passive Intranasal Monoclonal |
| | | | Antibody Prophylaxis Against Murine |
| | | | Pneumocystis Carinii Pneumonia,” Infect. Immun. |
| | | | 70: 1069-74 (2002), which is hereby incorporated |
| | | | by reference in its entirety |

(Gigliotti et al., “Passive Intranasal Monoclonal Antibody Prophylaxis Against Murine Pneumocystis Carinii Pneumonia,” Infect. Immun. 70: 1069-74 (2002); Gigliotti et al., “Development of Murine Monoclonal Antibodies to Pneumocystis Carinii,” J. Infect. Dis. 154: 315-22 (1986); Wells et al., “Epitope Mapping of a Protective Monoclonal Antibody Against Pneumocystis Carinii With Shared Reactivity to Streptococcus Pneumoniae Surface Antigen PspA,” Infect. Immun. 72: 1548-56 (2004), which are hereby incorporated by reference in their entirety). Several Pneumocystis-specific IgG and IgM antibodies were pooled and mice were injected i.p. with this anti-Pneumocystis antibody every three days after immune reconstitution. Control mice were given either phosphate buffered saline (PBS) or non-specific rat immunoglobulin (Jackson ImmunoResearch Laboratories, Inc., West Grove, Pennsylvania). Sulfasalazine (Ssz; Sigma, St. Louis, Missouri) was prepared fresh daily and given via intraperitoneal injections at a dose of 200 milligrams per kilogram (mg/kg) body weight as described (Wang et al., “Immune Modulation With Sulfasalazine Attenuates Immunopathogenesis but Enhances Macrophage-Mediated Fungal Clearance During Pneumocystis Pneumonia,” PLoS Pathog. 6: e1001058 (2010), which is hereby incorporated by reference in its entirety). Each group within an experiment contained 6-8 mice and each experiment was replicated 1-2 times. For the experiments assessing the effect of treatment with anti-Pneumocystis monoclonal antibody on macrophage phenotype, control mice received an equivalent amount of an IgG mouse monoclonal antibody to endotoxin plus an IgM mouse monoclonal antibody to the Haemophilus influenzae type b capsular polysaccharide.

Physiologic Assessment of Pneumocystis Infection.

Lung resistance and compliance were measured on living ventilated mice using a Harvard rodent ventilator (Harvard Apparatus, Southnatic, Mass.) and a whole body plethysmograph (BUXCO Electronics Inc., Wilmington, N.C.) as previously described (Wang et al., “Immune Modulation With Sulfasalazine Attenuates Immunopathogenesis but Enhances Macrophage-Mediated Fungal Clearance During Pneumocystis Pneumonia,” PLoS Pathog. 6:e1001058 (2010), which is hereby incorporated by reference in its entirety). Pulmonary function was normalized to body weight. Biosystems XA software package (BUXCO Electronics Inc., Wilmington, N.C.) was used to collect and analyze data. Bronchoalveolar lavage (BAL) was performed on whole lung with 4 milliliters (mL) of 1× Hank's balanced salt solution (HBSS).

Determination of Pneumocystis Lung Burden Using Quantitative Real-time PCR. Pneumocystis lung burden was quantified using real-time polymerase chain reaction (qPCR) using Pneumocystis kexin gene primers and a BioRad CFX96 Real-Time PCR Detection System (BioRad, Hercules, Calif.) as described (Lee et al., “Molecular Characterization of KEX1, a Kexin-Like Protease in Mouse Pneumocystis Carinii,” Gene 242:141-50 (2000), which is hereby incorporated by reference in its entirety).

Cytokine Analysis by ELISA with BAL and Lung Homogenate Supernatant.

Protein concentration was measured with DuoSet (R&D, Minneapolis, Minn.) for TNFα, IFNγ, monocyte chemoattractant protein (MCP)-1, IL-5, IL-17, IL-10, and IL-4 per manufacturer instruction. Enzyme-linked immunosorbent assays (ELISA) were performed on cell-free supernatant from BAL fluid and lung homogenate supernatant to measure cytokines and chemokines.

Flow Cytometric Analysis and Image Stream.

Fluorescence-activated cell sorting (FACS) analysis was completed for the following markers with monoclonal antibodies for CD4, CD8, CD11c, GR-1, DX-5, and CD3 (BD Biosciences, San Diego, Calif.). Image Stream analysis was used to evaluate the number of Pc organisms inside alveolar macrophages marked with CD11c monoclonal antibodies (Wang et al., “Immune Modulation With Sulfasalazine Attenuates Immunopathogenesis but Enhances Macrophage-Mediated Fungal Clearance During Pneumocystis Pneumonia,” PLoS Pathog. 6:e1001058 (2010), which is hereby incorporated by reference in its entirety). Pc organisms were identified using a pool of IgG and IgM specific anti-Pc antibodies. Image Stream data were collected using multispectral imaging flow cytometer (Amnis Corporation, Seattle, Wash.). Data was analyzed using the Image Stream Data Exploration and Analysis Software (IDEAS, Amnis Corporation, Seattle, Wash.).

Effect of Antibody Administration During Pcp on Alveolar Macrophage Phenotype.

For this experiment the Pneumocystis-infected mice were treated as above except that they received only 2 doses of specific antibody, nonspecific antibody or sulfasalazine on days 1 and 3 and then sacrificed later in the day on day 3. BALF aliquots containing 5×10⁴ cells in HBSS were cytospun onto glass slides, fixed with 3% paraformaldehyde in PBS for 15 minutes at room temperature; rinsed 3× in PBS; and permeabilized with 0.02% Triton X-100 in PBS for 15 minutes at room temperature. The slides were then rinsed 3× in PBS, blocked with species specific 5% normal serum (normal goat serum, MP Biomedical; normal donkey serum, EMD Millipore) in 0.05% Tween-20 in PBS (PBS-T) for 1 hr at room temperature and rinsed 3× in PBS-T. Alveolar macrophages were identified by staining with fluorescein labeled primary antibody to CD11c. Staining with antibody to iNOS was used to identify M1 macrophages while staining with antibody to YM-1 was considered a marker for M2 macrophages (Abcam and Stemcell).

DAPI (Molecular Probes) diluted 1:36 in PBS for 5 minutes at room temperature then rinsed 3 times in PBS was used to identify cellular nuclei.

Statistical Analysis.

Two way analysis of variance and multiple t-tests were completed using GraphPad Prism 6.05 software (GraphPad Software Inc., La Jolla, Calif.).

Results

Specific Anti Pneumocystis Antibody Reduced the Severity of Pneumocystis Infection and this Effect was Enhanced by Sulfasalazine.

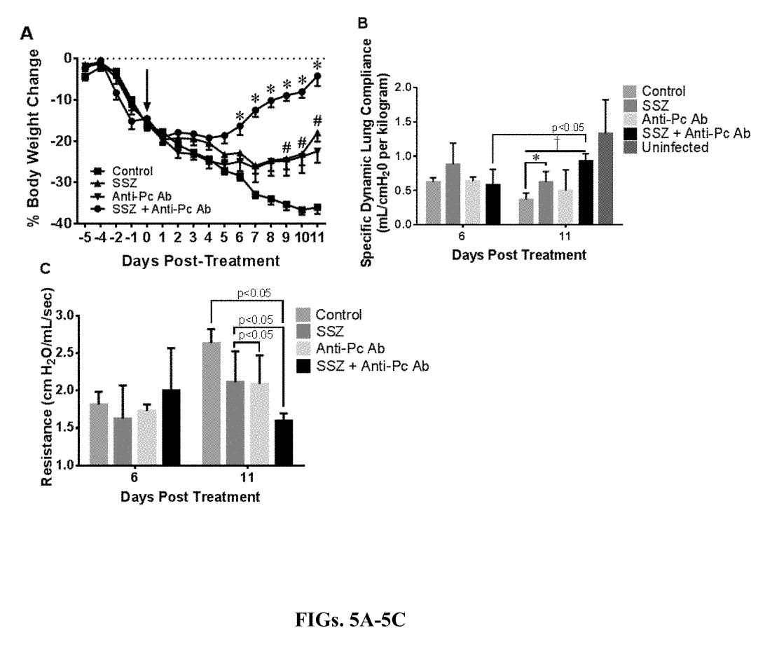

Mice displaying obvious clinical signs of Pcp-related Immune Reconstitution inflammatory Syndrome (IRIS) were treated with passive anti-Pneumocystis antibody, SSZ, or a combination of both. Mice treated with Pneumocystis-specific passive antibody or SSZ demonstrated reduced severity of Pcp as demonstrated by accelerated recovery of body weight and better pulmonary function measurements (FIGS. 5A-5C). The beneficial effects of individual treatments on Pcp were markedly enhanced by combination treatment with both specific anti-Pneumocystis antibody and sulfasalazine (FIGS. 5A-5C). Mice treated with the combination therapy began to recover weight by day 3 post-treatment compared to untreated mice with Pcp-IRIS, by day 11 post-treatment (FIG. 5A). The combination therapy group also had greater than 39% higher lung compliance (FIG. 5B) and 62% reduced lung resistance compared to untreated mice (FIG. 5C). These disease parameters have been shown to be reliable indicators of the severity of Pcp-IRIS, in this model, thereby lending credence to the physiologic significance of the observed results.

Specific Anti Pneumocystis Antibody Treatment Reduced the Lung Pneumocystis Burden and this Effect is Enhanced by the Addition of Sulfasalazine.

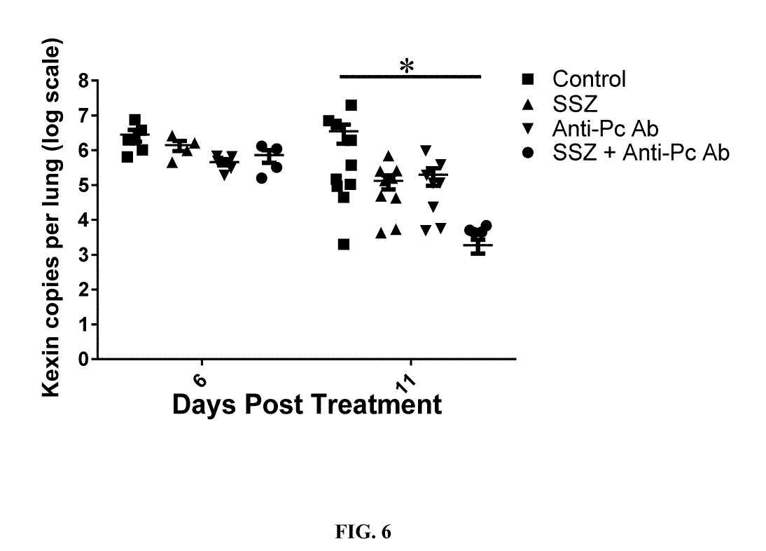

At day 11 post-treatment the specific anti-Pneumocystis antibody treated group had a reduction in lung Pneumocystis burden relative to control group (FIG. 6). As previously demonstrated (Wang et al., “Immune Modulation With Sulfasalazine Attenuates Immunopathogenesis but Enhances Macrophage-Mediated Fungal Clearance During Pneumocystis Pneumonia. PLoS Pathog. 6:e1001058 (2010), which is hereby incorporated by reference in its entirety), the lung Pc burden reduction was on the order of 1-2 logs. There was a significant enhancement in the clearance of Pc from the lung by combining treatment with antibody and SSZ that resulted in an undetectable lung Pc burden on day 11 post-treatment as measured by PCR.

Specific Anti Pc Antibody Treatment Increased the Uptake of Pc by Macrophages.

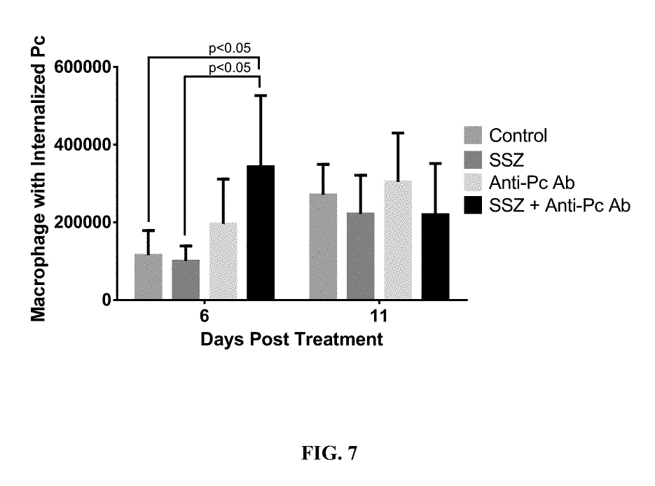

It was hypothesized that the effects of antibody on the progression of Pcp likely involved an interaction with alveolar macrophages exposed to specific anti-Pc antibody. To quantitate the Pc uptake by alveolar macrophages, an imaging flow cytometer (ImageStream) was used. A more rapid influx of alveolar macrophages into the lung at day 6 post-treatment was noted relative to control group, and this effect was significantly enhanced with addition of sulfasalazine treatment to specific anti-Pc antibody treatment (FIG. 7). By 11 days post-treatment the non-opsonized groups displayed “catch-up” in their ability to ingest Pc.

To test the effect of immune modulation, by administration of sulfasalazine or passive immunization, on macrophage phenotype during response to Pneumocystis, these agents or control nonspecific antibody were administered and alveolar macrophages were harvested after 3 days. As previously reported, administration of sulfasalazine resulted in an increase of the proportion of M2 type macrophages in the alveolar space (Wang et al., “Immune Modulation With Sulfasalazine Attenuates Immunopathogenesis but Enhances Macrophage-Mediated Fungal Clearance During Pneumocystis Pneumonia,” PLoS Pathog. 6:e1001058 (2010), which is hereby incorporated by reference in its entirety). Administration of antibody specific for Pneumocystis resulted in an approximately 40 fold increase in the proportion of M2 alveolar macrophages present in the lung in response to Pcp compared to those mice receiving nonspecific immunoglobulin.

TABLE 3
Effect of Administration of Anti-Pneumocystis Antibody or Sulfasalazine
on the Phenotype of Alveolar Macrophages in the Lung

Phenotype

| Treatment Day 3 | M1* | M2* |
|---|---|---|
| NS Ab | 290 | 10 |
| SSZ | 200 | 100 |
| Pc Mab pool | 125 | 126 |
| SSZ + Pc Mab pool | 125 | 175 |

*Per 300 macrophages counted

### DISCUSSION

Pcp is characterized by decreased pulmonary compliance leading to hypoxia, respiratory failure, and ultimately death. It is now clear that inflammatory injury to the lung is a crucial component of the pathogenesis of Pcp (Gigliotti and Wright, “Immunopathogenesis of Pneumocystis Carinii Pneumonia,” Expert Rev. Mol. Med. 7:1-16 (2005); Limper et al., “Pneumocystis Carinii Pneumonia. Differences in Lung Parasite Number and Inflammation in Patients With and Without AIDS,” Am. Rev. Respir. Dis. 140:1204-9 (1989); Hahn and Limper, “The Role of Inflammation in Respiratory Impairment During Pneumocystis Carinii Pneumonia,” Semin. Respir. Infect. 18:40-7 (2003), which are hereby incorporated by reference in their entirety). It has been shown that the depletion of CD8 T lymphocytes, which are critical to the initiation and maintenance of the damaging pulmonary inflammation, results in marked improvement in pulmonary function (Bhagwat et al., “Anti-CD3 Antibody Decreases Inflammation and Improves Outcome in a Murine Model of Pneumocystis Pneumonia,” J. Immunol. 184:497-502 (2010); Wright et al., “Immune-Mediated Inflammation Directly Impairs Pulmonary Function, Contributing to the Pathogenesis of Pneumocystis Carinii Pneumonia,” J. Clin. Invest. 104:1307-17 (1999), which are hereby incorporated by reference in their entirety). Another approach to protect the lungs from irreversible injury would be to interrupt various pro-inflammatory pathways (Gigliotti and Wright, “Immunopathogenesis of Pneumocystis Carinii Pneumonia,” Expert Rev. Mol. Med. 7:1-16 (2005); Bello-Irizarry et al., “The Alveolar Epithelial Cell Chemokine Response to Pneumocystis Requires Adaptor Molecule MyD88 and Interleukin-1 Receptor but not Toll-Like Receptor 2 or 4,” Infect. Immun. 80:3912-20 (2012); Wang et al., “Decreased Inflammatory Response in Toll-Like Receptor 2 Knockout Mice is Associated with Exacerbated Pneumocystis Pneumonia,” Microbes Infect. 10:334-41 (2008); Wang et al., “Pneumocystis Stimulates MCP-1 Production by Alveolar Epithelial Cells Through a JNK-Dependent Mechanism,” Am. J. Physiol. Lung Cell Mol. Physiol. 292:L1495-1505 (2007), which are hereby incorporated by reference in their entirety). One way to pursue this approach, for example, would be to enhance anti-inflammatory pathways.

It is now recognized that macrophages can vary in their biologic characteristics based on how they are activated. CAM are pro-inflammatory or M1 phenotype, and they can be programmed by exposure to LPS, TNFα, and IFNγ. AAM are anti-inflammatory or M2 phenotype, and they can be programmed by exposure to IL-4/IL-13 or antigen-antibody complexes (Tarique et al., “Phenotypic, Functional and Plasticity Features of Classical and Alternatively Activated Human Macrophages,” Am. J. Respir. Cell Mol. Biol. 53(5):676-88 (2015); Arango Duque and Descoteaux, “Macrophage Cytokines: Involvement in Immunity and Infectious Diseases,” Front Immunol. 5: 491 (2014), which are hereby incorporated by reference in their entirety). Thus, treatments that bring about a shift in macrophage phenotype to an M2 or anti-inflammatory phenotype should facilitate resolution of Pcp.

The way antigen is presented to macrophages may affect macrophage phenotype. For example, presentation of antigen complexed with antibody results in activation of macrophages via anti-inflammatory M2 pathways. Therefore, it was hypothesized that since both specific anti-Pc antibody treatment and SSZ offered significant protection, combining the two treatment modalities might offer even more protection. This was in fact what was observed. While both specific anti-Pneumocystis antibody and SSZ produced some benefit in mice with Pcp, the combination of the two treatment modalities showed an enhanced effect. That the benefit noted in the experiment was due to the effect of specific anti-Pneumocystis antibodies and not to some non-specific immune modulatory effect from non-specific high-dose immunoglobulin is supported by the observation that the irrelevant antibody treated mice in the control group displayed none of the beneficial effects noted in the specific anti-Pneumocystis antibody treated group. An important feature of the experimental approach is that anti-Pneumocystis antibody and SSZ were not administered until the mice with Pcp infection became clinically symptomatic. This more closely mimics how patients with Pcp present for medical care. Additional studies will be necessary to determine whether there is a relationship between the specific antigen or antigens targeted for opsonization and the beneficial effects that were observed.

The precise role for antibody in the immune response to fungal infections is incompletely understood. Pcp is exacerbated by inflammatory damage when cellular immunity is restored, but it appears that this inflammatory insult responds to the administration of specific anti-Pneumocystis antibody. This effect was enhanced by the addition of the immune modulatory agent sulfasalazine.

In the present model, Pneumocystis infection created significant illness prior to starting treatment. For cryptococcal fungal infections there has been demonstration of modified outcome with passive administration of specific fungal antibodies to cryptococcal capsular glucuronoxylomannan prior to infection (Mukherj ee et al., “Monoclonal Antibodies to Cryptococcus Neoformans Capsular Polysaccharide Modify the Course of Intravenous Infection in Mice,” Infect. Immun. 62:1079-88 (1994); Mukherjee et al., “Protective Murine Monoclonal Antibodies to Cryptococcus Neoformans,” Infect. Immun. 60:4534-41 (1992), which are hereby incorporated by reference in their entirety). In that experiment monoclonal antibodies were given up to 24 hours prior to infection, and mice that received antibody had less cryptococcal fungal burden. Another study from the same group evaluated more specific groups of anti-cryptococcal antibodies and found that passive administration prior to infection decreased cryptococcal fungal burden except for a few antibodies that led to worse infection with increased mortality (Shapiro et al., “Immunoglobulin G Monoclonal Antibodies to Cryptococcus Neoformans Protect Mice Deficient in Complement Component C3,” Infect. Immun. 70:2598-604 (2002), which is hereby incorporated by reference in its entirety). In the same study, C3 complement deficient mice had improved protection with specific anti-cryptococcal antibodies compared to mice with normal complement demonstrating the role complement plays with antibody for protection against fungal pathogens. To evaluate protection of specific antibodies further, the group used a single anti-cryptococcus monoclonal antibody to test for protection against cryptococcus and found that mice developed acute lethal toxicity leading to hypotension and circulatory collapse in many of those mice (Lendvai and Casadevall, “Monoclonal Antibody-Mediated Toxicity in Cryptococcus Neoformans Infection: Mechanism and Relationship to Antibody Isotype,” J. Infect. Dis. 180:791-801 (1999), which is hereby incorporated by reference in its entirety).

The enhanced physiologic improvement and decreased lung Pneumocystis burden was seen without standard of care pneumocystis antibiotic treatment with trimethoprim-sulfamethoxazole (TMP-SMX). Future studies will need to evaluate how treatment with TMP-SMX affects the present findings.

As described herein, more rapid resolution of lung inflammation during Pcp was demonstrated via multiple mechanisms. There was less inflammation in groups treated with specific anti-Pneumocystis antibody as evidenced by decreased cell counts, and this effect was enhanced by the addition addition of sulfasalazine. This is likely due to alteration of inflammatory immune response. In a previous study, it was demonstrated that sulfasalazine reduced inflammation associated with Pc infection (Wang et al., “Immune Modulation With Sulfasalazine Attenuates Immunopathogenesis but Enhances Macrophage-Mediated Fungal Clearance During Pneumocystis Pneumonia,” PLoS Pathog. 6:e1001058 (2010), which is hereby incorporated by reference in its entirety). Sulfasalazine has been shown to have immune modulatory effects via blockage of inflammatory transcription factor nuclear factor kappa beta NF-κB (Wahl et al., “Sulfasalazine: A Potent and Specific Inhibitor of Nuclear Factor Kappa B,” J. Clin. Invest. 101:1163-74 (1998), which is hereby incorporated by reference in its entirety). In that in vitro study, sulfasalazine blocked transcription of NF-κB which led to decreased production of TNF-alpha. It has been previously demonstrated that Pc activates NF-κB production in alveolar epithelial cells (Wang et al., “Pneumocystis Carinii Activates the NF-KappaB Signaling Pathway in Alveolar Epithelial Cells,” Infect. Immun. 73:2766-77 (2005), which is hereby incorporated by reference in its entirety). Enhancement of specific anti-Pc antibody effect in the study with addition of sulfasalazine is likely due to sulfasalazine effects on NF-κB that lead to downstream decrease in inflammation. This is supported by lower cell counts and improved resolution in groups treated with specific anti-Pneumocystis antibody and sulfasalazine compared to no treatment groups. Nonspecific antibody treatment has been shown to decrease inflammatory parameters in diseases such as Kawasaki disease (Lee et al., “High-Dose Intravenous Immunoglobulin Downregulates the Activated Levels of Inflammatory Indices Except Erythrocyte Sedimentation Rate in Acute Stage of Kawasaki Disease,” J. Trop. Pediatr. 51:98-101 (2005), which is hereby incorporated by reference in its entirety) and alter immune responses in several diseases including Guillain-Barre disease and systemic lupus erythematosus (Siberil et al., “Intravenous Immunoglobulin in Autoimmune and Inflammatory Diseases: More Than Mere Transfer of Antibodies,” Transfus. Apher. Sci. 37:103-7 (2007), which is hereby incorporated by reference in its entirety). In the present model, non-specific antibody had no demonstrable effect on the resolution of Pcp. As described herein, groups treated with specific anti-Pc antibody demonstrated less inflammatory cell influx and improved resolution of PCP.

In addition to decreased inflammation, specific anti-Pneumocystis antibody treatment was associated with decreased lung Pneumocystis burden, and this effect was enhanced with addition of sulfasalazine. One explanation is phagocytosis of Pneumocystis is more efficient with addition of specific anti-Pc antibody enhanced with sulfasalazine. There were significantly increased number of alveolar macrophages with internalized Pc in mice treated with combination specific anti-Pneumocystis antibody and sulfasalazine at early time points during the course of infection compared to sulfasalazine alone or specific anti-Pneumocystis antibody alone. One reason is due to increased opsonization of Pneumocystis with specific anti-Pneumocystis antibody. Specific antifungal antibodies have been shown to increase phagocytosis in a Candida fungal infection (Wellington et al., “Enhanced Phagocytosis of Candida Species Mediated by Opsonization With a Recombinant Human Antibody Single-Chain Variable Fragment,” Infect. Immun. 71:7228-31 (2003), which is hereby incorporated by reference in its entirety). Likely specific anti-Pc antibodies help opsonize Pneumocystis in a similar manner, and this effect is enhanced with addition of sulfasalazine.

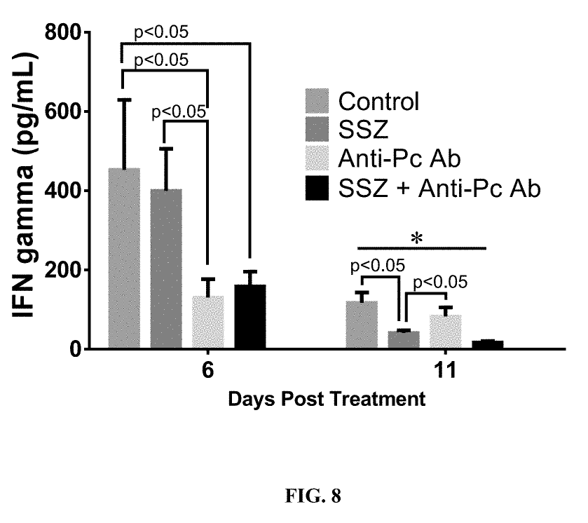

Treatment with specific anti-Pc antibody altered immune response based on the ELISA data investigating different cytokines. Mice treated with specific anti-Pc antibody had decreased TNF-alpha and IFN-gamma levels in lungs relative to no treatment (FIG. 8). Antibodies have been shown to increase opsonization, but specific antifungal antibody treatment has not been demonstrated to alter these types of cytokines.

It is possible that the additive effects of SSZ on inflammation and pathogen clearance in the present model could be caused by the elicitation of a pro-resolution phenotype in alveolar macrophages. Although still a matter of some debate (Martinez and Gordon, “The M1 and M2 Paradigm of Macrophage Activation: Time for Reassessment,” F1000Prime Rep. 6:13 (2014), which is hereby incorporated by reference in its entirety), the ability of macrophages to differentiate into transcriptionally distinct functional states (e.g., M1, M2a, M2b etc) in response to different cytokines and other environmental cues is well-documented (Labonte et al., “The Role of Macrophage Polarization in Infectious and Inflammatory Diseases,” Mol. Cells 37(4):275-85 (2014); Roszer, “Understanding the Mysterious M2 Macrophage Through Activation Markers and Effector Mechanisms,” Mediators of Inflamm. 2015:816460 (2015), which are hereby incorporated by reference in their entirety). While M1 macrophages are associated with pro-inflammatory and immune activating stimuli (i.e., IFNγ, TNF, LPS), alternatively activated (M2) macrophages are skewed toward promoting the resolution of immune responses by producing IL-10 and suppressing pro-inflammatory mediators like TNF and IL-12 (Martinez and Gordon, “The M1 and M2 Paradigm of Macrophage Activation: Time for Reassessment,” F1000Prime Rep. 6:13 (2014), which is hereby incorporated by reference in its entirety). Interestingly, M2-polarized macrophages have also been reported to show increased levels of the B-glucan receptor Dectin-1 (Taylor et al., “Macrophage Receptors and Immune Recognition,” Ann. Rev. Immunol. 23:901-44 (2005); Gales et al., “PPARy Controls Dectin-1 Expression Required for Host Antifungal Defense Against Candida albicans,” Plos Pathogens 6(1):e1000714 (2010), which are hereby incorporated by reference in their entirety), increased complement-mediated phagocytosis (Freeman and Grinstein, “Phagocytosis: Receptors, Signal Integration, and the Cytoskeleton,” Immunological Reviews 262(1):193-215 (2014), which is hereby incorporated by reference in its entirety) and increased phagosomal degradation (Balce et al, “Alternative Activation of Macrophages by IL-4 Enhances the Proteolytic Capacity of Their Phagosomes Through Synergistic Mechanisms,” Blood 118(15):4199-208 (2011), which is hereby incorporated by reference in its entirety). Consistent with this idea, the bioactive metabolite of SSZ, 5-ASA, is an activating ligand for the pro-M2 transcription factor PPARγ (Rousseaux et al, “Intestinal antiinflammatory effect of 5-aminosalicylic acid is dependent on peroxisome proliferator-activated receptor-gamma,” J. Exp. Med. 201(8):1205-15 (2005), which is hereby incorporated by reference in its entirety). Thus it is possible that anti-Pc and SSZ together elicit an M2-like program in alveolar macrophages that could result not only in suppressing inflammation in the lung, but could augment the phagocytic clearance of Pc by alveolar macrophages either directly (via Dectin-1 (Steele et al., “Alveolar Macrophage-mediated Killing of Pneumocystis carinii f. sp. muris Involves Molecular Recognition by the Dectin-1 Beta-glucan Receptor,” J. Exp. Med. 198(11):1677-88 (2003), which is hereby incorporated by reference in its entirety) or indirectly (increased antibody-mediated clearance).

More studies have started to look at alteration of macrophage phenotype to improve inflammatory conditions (Edwards et al., “Biochemical and Functional Characterization of Three Activated Macrophage Populations,” J. Leukoc. Biol. 80:1298-1307 (2006); Bystrom et al., “Resolution-Phase Macrophages Possess a Unique Inflammatory Phenotype That is Controlled by cAMP,” Blood 112:4117-27 (2008); Tatano et al., “Unique Macrophages Different From M1/M2 Macrophages Inhibit T Cell Mitogenesis While Upregulating Th17 Polarization,” Sci. Rep. 4:4146 (2014); Ferrante et al., “The Adenosine-Dependent Angiogenic Switch of Macrophages to an M2-Like Phenotype is Independent of Interleukin-4 Receptor Alpha (IL-4Ralpha) Signaling,” Inflammation 36:921-31 (2013), which are hereby incorporated by reference in their entirety). There are inflammatory and anti-inflammatory phenotypes. It has been shown that sulfasalazine treatment of Pc-infected mice alters alveolar macrophage phenotype to a more anti-inflammatory phenotype and improves PCP resolution (Wang et al., “Immune Modulation With Sulfasalazine Attenuates Immunopathogenesis but Enhances Macrophage-Mediated Fungal Clearance During Pneumocystis Pneumonia,”PLoS Pathog. 6:e1001058 (2010), which is hereby incorporated by reference in its entirety). One possibility is that specific anti-Pc antibody alters alveolar macrophages to a more phagocytic and less inflammatory phenotype. The macrophage Pc internalization data and ELISA data support this possibility. Increased IL-10 is associated with anti-inflammatory macrophage phenotype, and mice treated with specific anti-Pc antibody and sulfasalazine showed an increase in IL-10 relative to no treatment. These effects are enhanced with addition of sulfasalazine which is known to alter macrophage phenotype. Future studies could be completed to examine alveolar macrophage phenotype alteration under specific anti-Pc treatment.

In conclusion, this is the first time specific anti-Pc antibody treatment has demonstrated this degree of improved resolution and decreased inflammation in PCP infection with enhancement via immune modulation with sulfasalazine. Further studies need to be completed to evaluate alteration of macrophage phenotype by specific anti-Pc antibodies and to look at more detailed mechanistic studies.

### Example 3—Administration of a Pool of Mixed Monoclonal Antibodies to Pneumocystis Results in Amelioration of Pneumocystis Pneumonia

Materials and Methods

Using the mouse model of Immune Reconstitution Inflammatory Syndrome (IRIS), SCID mice with pneumocystis pneumonia were reconstituted with normal splenocytes five to six weeks after onset of pneumocystis infection. To more realistically mimic the human condition, mice were monitored for the onset of symptoms, notably weight loss and increased respiratory rate. It has been demonstrated that loss of 10% of body weight is a reliable marker of moderate to severe pneumocystis pneumonia. Symptomatic mice were given the following antibodies: Group A (six mice) were given an irrelevant IgG and IgM monoclonal antibody and served as the negative control for this experiment, Group B (seven mice) received three IgM monoclonal antibodies, two which recognized PCA-1 (4F11 and 1G4) and one bound to the unrelated antigen GpA (5E12). Group C (seven mice) received monoclonal antibody 4F11 (G1), which is the IgG switch variant of the monoclonal antibody 4F11.

Mice received two doses of antibody per week for two weeks for a total of four doses. On day 14 the mice were sacrificed and the effect of antibody treatment on the pneumocystis pneumonia was defined.

At various times, as indicated, measurements of arterial oxygenation and dynamic lung compliance were done. The lungs of some mice were removed after dynamic compliance measurements were taken. These lungs were homogenized and aliquots were stained with Gomori's methenamine-silver to confirm and quantify the P. carinii burden.

Pulmonary compliance was measured in live mice using a method described previously, with modifications (Bergmann et al., “Lung Mechanics in Orally Immunized Mice After Aerosolized Exposure to Influenza Virus,” Respiration 46:218-221 (1984), which is hereby incorporated by reference in its entirety). Mice were anesthetized with 0.13 mg sodium pentobarbital per gram of body weight administered intraperitoneally. Mice were then surgically cannulated through the trachea with an 18-gauge cannula inserted 3 mm into an anterior nick in the exposed trachea and immediately placed on a Harvard rodent ventilator (Harvard Apparatus, South Natick, Mass., USA) at a respiratory rate of 150 strokes per minute. To ensure that the mice tolerated the procedure, they were examined for spontaneous respirations before proceeding further. The thorax was opened to equalize airway and transpulmonary pressure. The animal was placed in a pressure plethysmograph and ventilated at 2.5 Hz with a tidal volume of 0.01 milliliter per gram of body weight. Signals for airway pressure and volume were passed through an analogue-to-digital converter and used to calculate pulmonary compliance using the method of Amdur and Mead (Amdur and Mead, “Mechanics of Respiration in Unanesthetized Guinea Pigs,”Am. J. Physiol. 192:364-368 (1958), which is hereby incorporated by reference in its entirety). Compliance was normalized for either body weight or length, as indicated.

Results

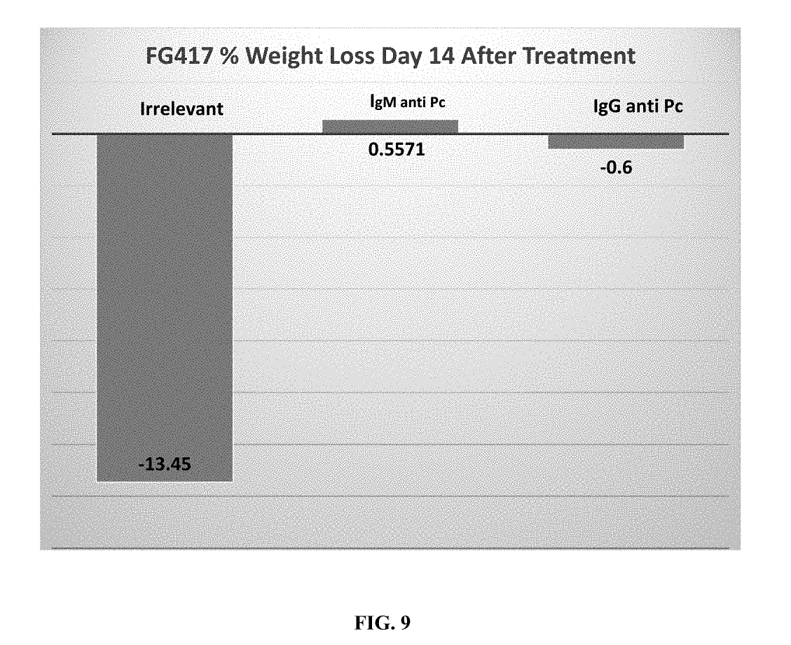

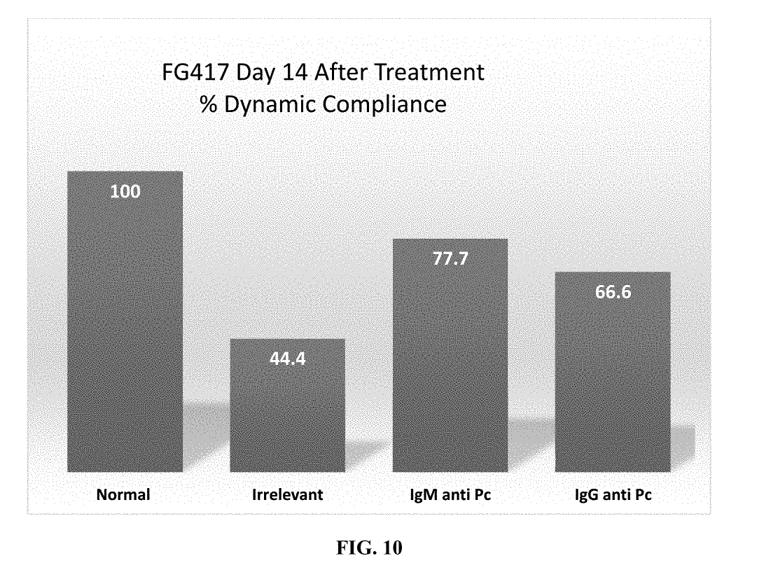

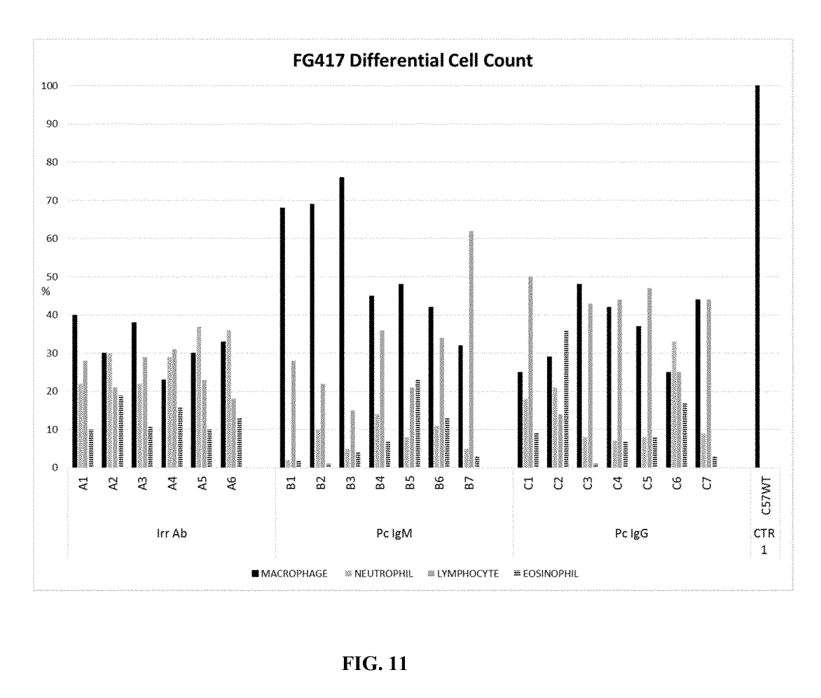

Mice which received cross reactive monoclonal antibodies had significantly improved pulmonary function including higher compliance (FIG. 10) and clinically significant less weight loss (FIG. 9) and fewer inflammatory cells in their lungs (FIG. 11). Specifically, the observed reduction in neutrophils indicates the resolution of lung injury. These results were also seen in Group C mice which received only the IgG cross reactive monoclonal antibody 4F11(G1).

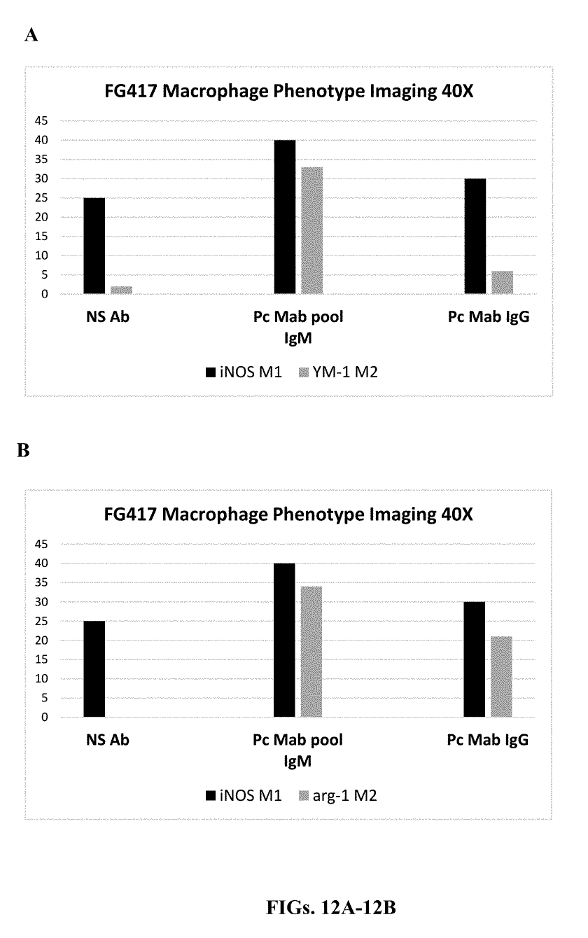

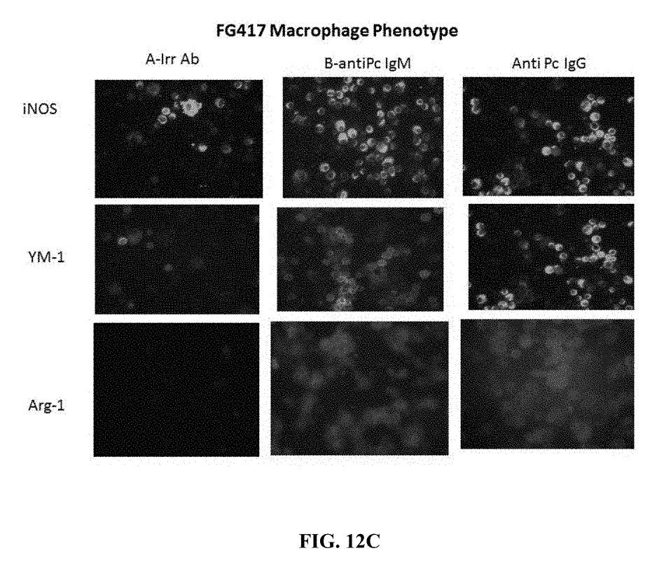

Administration of passive antibody which binds to PCA1 resulted in marked psychologic improvement in mice with pneumocystis pneumonia, even though they did not receive antibiotic treatment for their pneumocystis pneumonia. In other experiments this treatment also resulted in a shift in macrophage phenotype to that of M2 macrophages, which is a resolution macrophage phenotype (FIGS. 12A-12C). Because these monoclonal antibodies react with pneumocystis of human origin, these results strongly support the potential use of these antibodies as adjunct treatment for pneumocystis pneumonia.

What is claimed:

1. A method of treating Pneumocystis pneumonia infection in a subject, said method comprising:
administering to a subject having a Pneumocystis pneumonia infection one or more antibodies that bind specifically to a Pneumocystis cross-reactive antigen 1 (PCA1) protein under conditions effective to treat the Pneumocystis infection in the subject.

2. The method according to claim 1, wherein the one or more antibodies bind to one or more epitopes in SEQ ID NO:1.

3. The method according to claim 1, wherein the one or more antibodies are humanized.

4. The method according to claim 1, wherein the PCA1 is murina.

5. The method according to claim 1 further comprising:
repeating said administering.

6. The method according to claim 1, wherein said administering is carried out orally, by inhalation, by intranasal instillation, topically, transdermally, parenterally, subcutaneously, by intravenous injection, by intra-arterial injection, by intramuscular injection, intraplurally, intraperitoneally, by intracavitary or intravesical instillation, intraocularly, intraventricularly, intralesionally, intraspinally, or by application to mucous membranes.

7. The method according to claim 1, wherein the subject is a mammal.

8. The method according to claim 7, wherein the mammal is an ungulate, a non-human primate, a rabbit, a ferret, a rat, or a mouse.

9. The method according to claim 1 further comprising:
administering to the subject an antibiotic agent effective against Pneumocystis and/or immunomodulatory agent.

10. The method according to claim 9, wherein an antibiotic agent is administered, and the antibiotic agent is selected from the group consisting of co-trimoxazole, pentamidine, primaquine, and trimethoprim plus sulfamethoxazole.

11. The method according to claim 9, wherein an immunomodulatory agent is administered, and the immunomodulatory agent is an anti-inflammatory.

12. The method according to claim 11, wherein the anti-inflammatory is sulfasalazine.

13. The method according to claim 1, wherein the one or more antibodies comprise monoclonal antibody 4F11.

14. The method according to claim 1, wherein the one or more antibodies comprise monoclonal antibody 1G4.

15. The method according to claim 1 further comprising:
administering to the subject a Pneumocystis protein or polypeptide comprising the amino acid sequence of SEQ ID NO:1 or antigenic fragment thereof.

16. A method of treating a subject at risk for Pneumocystis pneumonia infection, said method comprising:
administering to a subject at risk for Pneumocystis pneumonia infection a Pneumocystis protein or polypeptide comprising the amino acid sequence of SEQ ID NO:1 under conditions effective to prevent the Pneumocystis infection.

17. The method according to claim 16 further comprising:
repeating said administering.

18. The method according to claim 15, wherein said administering is carried out orally, by inhalation, by intranasal instillation, topically, transdermally, parenterally, subcutaneously, by intravenous injection, by intra-arterial injection, by intramuscular injection, intraplurally, intraperitoneally, by intracavitary or intravesical instillation, intraocularly, intraventricularly, intralesionally, intraspinally, or by application to mucous membranes.

19. The method according to claim 16, wherein the subject is a mammal.

20. The method according to claim 19, wherein the mammal is an ungulate, a non-human primate, a rabbit, a ferret, a rat, or a mouse.

21. The method according to claim 16 further comprising:
administering to the subject an antibiotic agent effective against Pneumocystis.

22. The method according to claim 21, wherein the antibiotic agent is selected from the group consisting of co-trimoxazole, pentamidine, primaquine, and trimethoprim plus sulfamethoxazole.

23. The method according to claim 16 further comprising:
administering to the subject one or more antibodies that bind specifically to a Pneumocystis cross-reactive antigen 1 (PCA1) protein.

24. The method according to claim 23, wherein the one or more antibodies bind to one or more epitopes in SEQ ID NO:1.

25. The method according to claim 23, wherein the one or more antibodies are humanized.

26. The method according to claim 23, wherein the one or more antibodies comprise monoclonal antibody 4F11.

27. The method according to claim 23, wherein the one or more antibodies comprise monoclonal antibody 1G4.

28. The method according to claim 23, wherein the PCA1 is murina.

29. An isolated protein or polypeptide comprising the amino acid sequence of SEQ ID NO:1.

30. A pharmaceutical composition comprising:
the isolated protein or polypeptide according to claim 28 and
a pharmaceutically acceptable carrier.


SEQUENCE LISTING (5 sequences)

SEQ ID NO: 1 - protein, 1099 aa, mouse
MFFLRIIFIFIFLKISYAENTDKLSDFEKKYPELYQANPHALKLEALKSGFSGKSVKKGLGVFHIGNLGHYRDHKPVILHVIMGLTVGLAECRGTLAERCKVIKALGNPITQYCNKPYDTCQDYFDARNYLLPMKDQLKNPHAHHDACRTILLNCLFFKHRNYITSDCVPLVALCYLRVRQNFVEAIMTEALRGEINTKGAAAAMKKVCEKIGHESPDLLHLCFKTTVLEKPKRSNKQYIEDVKSRIRTVSTGNCRQVLEECYFNVLDYPDIYQSCRNFRRFCSEIGVVYTPVDSTFDLFQKPLSAEKLLIDTSSKISEDLGLGFSKYVQKKSSNLEIAAYLVNKTWVYDNDCRNKLKELCLHIASLPLTKQLCTLAHDRNSKLCRDFYNSIGTECYSLYYEFKNVGLLYNYTYRLSRDQCSKYVERCLFLREQYAYWNSLDTCANVFSSCYKEDMDFSAKLDLLNRIKDKIVVPKGNTRYFVELLCKSYIVAECSASDLMFKSYALMEACLHPERICRELKNHFSEESRKLENKLRSILKPTYYECKDLGQKCNSGFYFDGDIEAQCNHFKKRCQDKQERLKLINHIVDSSALYLANEVQCRTYFDSFCGANVKQEFKQICNKGANGICPDIIDDSKEHCAHLINHLTSLGISSSSASLPLDYCDSAINYCNSLSKFCTESKRQCDSVISFCTSESKKTDEYGSFIDQYPAAAANATKCKVTLKELCQDSSKKDSYSTLCAYNKDGYTEICKNLRNFIEKACENLRIHLHTYDTNSLNTNKGSAQDRCTYIRNLYFKFKNICLLVDPFYDLSPIITQECKTNISEPALPDKDPQPTSSPQPKPRPRPRPQPQPHPHPKPQPQPTPEPQPQPAPEPRPQPTSKPRPQPTSKPRPQPTPEPRPLPVPGPGPLPVPGPRPQPQPQPQPQPQPQPQPQPQPQPQPQPQPQPQPQPKPQPPSQSTSESASQSKPKPTTQTKPSPRPHPKPVPKPSSIDTGPSKSDSSFIFTVTKTITKISETEKPSTKPSVKPTSTKTTSKPSTKPSTKPSVKPASTKTTSESEKPTLEEVPETKGNGVRVIGFEGLQLLSMIVAIIIGIWIM

SEQ ID NO: 2 - DNA, 3300 bp, mouse
atgtttttcttaagaatcatctttatatttatttttttaaaaatatcatatgcagaaaacacagataaactctcagatttcgaaaaaaaatatccagaattatatcaagcaaatccacatgctttaaaactggaagcattgaaaagcggattttcaggcaaatctgtaaaaaaaggattgggtgtttttcatatagggaatcttggtcattatagagatcataaaccagttatattgcatgtaattatgggattaactgttggactcgcagagtgtcgcgggacactcgccgaaagatgtaaagtcataaaagccctaggaaatccaataacacaatattgcaataaaccatatgatacatgccaagattattttgacgctcgaaattacttactccctatgaaagatcaattaaaaaacccacacgcccatcatgatgcatgcagaacgattttgctaaattgcctcttttttaaacatcgtaattatattacttccgattgtgttcctttggtagcattatgttatttgcgggttcgtcaaaactttgtagaagcaattatgaccgaagcattaagaggggaaattaatactaagggtgctgctgcagcaatgaaaaaagtatgtgaaaaaattggacatgagagtccggacttgcttcatttatgttttaagaccactgtattagaaaaacctaaaaggtctaataaacagtatattgaagatgttaagtcaagaataaggacagtttcgactggaaattgccgtcaggttttggaagaatgctattttaatgttctagattatccagatatttatcaatcatgtaggaattttcgacgattctgttcagaaataggagttgtatatactccagtcgattccacttttgatttatttcagaagcccctttctgcagaaaagttactaattgatacttcttcaaaaatctcagaagacttaggtcttggtttttctaaatatgtacaaaaaaaatcaagcaatcttgagattgcggcatatttagttaataagacttgggtctatgataatgattgcagaaataaattaaaagaactatgtctgcatattgcttctctaccgcttacaaaacaactatgcacattagcacatgatagaaattcgaaactctgtagggatttttataactctattgggactgaatgctattctttatattatgaatttaagaatgttggattattatacaattatacttatcgtctttcaagagatcaatgctctaaatatgtagaaagatgtctttttcttagggagcaatatgcttattggaattctctagatacttgtgctaatgtattttcttcatgttataaagaagatatggatttttcagccaaattagatcttctaaataggataaaagataagattgtagttccaaaaggaaacacgaggtattttgtagagttattgtgtaaaagctatattgtcgccgaatgcagcgccagtgatttaatgttcaaatcttatgctcttatggaagcctgtcttcacccagaaaggatctgtagagaattaaaaaatcatttttccgaagaatctaggaaattagaaaataaattaaggagtattttaaaacccacatattatgaatgcaaagatctaggacaaaagtgcaactctggattttattttgatggagatatagaagctcaatgcaatcatttcaaaaaaagatgtcaagataaacaagagagactaaaattaattaatcatattgttgattcatctgctctttatctcgcaaatgaagtacaatgcagaacttatttcgacagtttttgtggtgcgaatgtaaaacaagaattcaaacaaatatgcaacaaaggagctaatggcatatgccctgatataatagatgattctaaagaacattgtgctcatttgattaatcatttaacatctcttggaatttcatcgtcttctgcttcacttccattggactattgcgactcagcgattaattactgtaattctctttcgaagttttgcacggaatcaaaacgacagtgcgattctgttatttctttctgcactagcgaatcaaaaaaaactgatgaatatggttcttttattgaccaatatcccgcggctgcagcaaatgcaaccaaatgcaaggtaactttgaaagagttatgccaagattcaagcaaaaaagactcttattcaacactatgtgcttataataaagatggttataccgaaatatgtaaaaacttaagaaatttcatagaaaaagcatgcgagaatttgagaattcatttacatacttatgatacaaactcactcaatacgaataaaggatctgctcaagatagatgcacttatataagaaatctttactttaaatttaaaaatatatgtttattggttgatcctttctatgacttatctcctattatcactcaagaatgtaaaaccaatatatccgaaccagcactgcctgataaggatcctcaacctacatcttcacctcagccaaaacctcggccaagacctcgacctcaacctcaacctcatccacatccaaaacctcagcctcagccgacgccagaacctcagcctcagccggcgccagaacctcgacctcagccgacgtcaaaacctcgacctcagccaacgtcaaaacctcgacctcagccgacgccagaacctcgacctctgccggtgccaggacctggacctctgccggtgccaggacctcgacctcaacctcaacctcaacctcaacctcagcctcaacctcaacctcagcctcaacctcaacctcagcctcagcctcagcctcagcctcagcctcaacctcagccgaagcctcaaccaccatctcagtcaacatcagaatcagcatcgcaatccaaaccaaaaccaacaacacaaacaaaaccgtcaccgagaccacacccaaagccggtgccaaaaccatcatcgatagacacaggaccatcaaaatcggattcaagcttcatttttacagtaacaaaaacaataacaaagatatcagaaacagaaaaaccatctacaaaaccatctgtgaaaccaacctctacaaagacaacatcaaaaccatctacaaaaccatctacaaaaccatctgtaaaaccagcctctacaaagacaacatcagaatcagaaaaaccaacattggaagaagttccagaaactaaagggaatggtgtaagagtaataggatttgaggggttacaattattatcaatgattgttgcaataataattgggatatggataatgtaa

SEQ ID NO: 3 - protein, 974 aa, pneumocystis jirovecii
MQLFLGTCIFLIFISIKRVFSEDDGLINDYSPLYEDDPLLSSILKSDEYHVQLYEQLKWIEHENLWQNYKDDYYYLATILNSYDLYYYCEAKLKEACIDIEDLSKRFQKICKKPQESCNNLYNKIIKKVKELVTIFSNGQKDGYDRCSKLLIECFFLEHHAPSYIGDECTTLKEYCYNKVRMGVAEIIVYDFLRGTSNDLDTCIRKLRNECQSINQRSPELFGLCINVKDICKKFILKSKKECQSSQISHPLKQISQEKCRTLLAKCYSILSDCPILRSQCRLLKMDCAEKGFFYDISENYNFGLLENPSTHMNRNGVEYAYGKLYNSGIHVTKIQNLSDLLIVNFLINKTTAVDNDCKVMLDKKCSSINYLSYIKPICNASSNNEYKICKDIYYKTKDYCYSLLKKFKESNQFWWYSDRPFSKTECEEYLSICYFIGEYFYYWMSYDTCKYVRVVCYQENLDAATNVILIKKLSGKFELKRDNRGIIALYRAMSDCKNALLEECGNFMYHSYPILYKCLHPKETCRNLTNLLDKNCERLEENLKAAIPNPEYTTCRQLKKECNELGPYSESTLKLCRNFKKICENVDKLMEFKLSILKKKNSVFKNNTTCFDYLNDYCSKNVASHICTNKNKSCTQMLDQISIHCEQLSFYLSYYKSSNGQINHINYGRCSQFLNYCKMLKETCSNLTSICFQVEQSCNKISTEKENVITLIKVVGKEIFNRSKCENRLDEICKNTITKKKMNELCTRVNDTCKILKLHLEKICDQLTLKIFNFLFTNSKSRIECQKLTLLCSSIASSCNETNVKVLPLCLNFTIVCSKLPEQPKPPKPPKPSEPSEPPELPEPPESPESPESPELPESSESSELPRPKPTLTNSTTILTSTQSSTTSLSTTSITKSSATRSSTTRSSATRSSTTRSTTRSTTRSTTRSLRPKPTISSDDRRIIGFGIRPRKLRGIELIWMIAGTILGMWIIV

SEQ ID NO: 4 - protein, 1099 aa, pneumocystis murina; identical to SEQ ID NO: 1

SEQ ID NO: 5 - protein, 1006 aa, pneumocystis carinii
MLFLKILFICLFARTYAKDPSKLSEFGREYPPLYQANSFISKIERTHSKWTEDYIRSKLYALAWQNSIDRNNDAFIFSSLIGAISNMNYCAEMLRKRCEVLKALGGKVTEYCVDPVRTCGFHFPVRQIRAALQSNLRKRYTTRTACTEYLRTCFFLKHKLSMATECTFLIGSCYLWVRKHVAEAILSEVMRGDLSKQTSAYSLRRACEVVGGGSPDLLRLCFENGNLLKKLRTGLQQEISALKTRLSLVSYRNCYYTLQQCYYYIADYYGIYEGCKELRRACEETGYSFKPPTFSPHMLEETIYLEDKIVSIASEVSSSLGLRFSSCVTRPSSDIEVAAYLVNKTWMYDYECTNKLQGVCQDISSIPYTRKLCSSLKNRDSKPCKDLYYSVRDESYSLYNALSDAGILRNTTQRLSLQQCDIFIERCSFLRTHYSYWNSLDTCLIVYSACYKEDIDQSAKIYLQTKLKKEMIVGDKFDESKCKEAVLKECRLENLMFRSYSLMEACFHSERFCQEIKKHLTQESKKLEEEVKKKKGSPSFYDCKELGQKCNSGLYFGSNVKNECKSFQEKCNEKEVYNQLLNHTLHLNASFLTSEDQCKTEFSKSNSNNHRFKEIIYKKDDPKVCKALLDMLKERCDNMEYVLITLKSSANSLSLTYCSYISTNCKLLSKACPKLKEHCDPVASACDTKSQELNEQSSFIDKHPMDVADSKKCKTKLQELCKNQEEKKNYSLLCARNDNHVRSCSDLKDFLEKACAALRSFLYSFLSGSKYSDKCSTLKELYFKFKKICLTVDKYFDLSLYAKEKCELDKEPEPEKPKPPTPKPPAPKPPSPPSPPSPPQPPQPPQPQPQPQPQPQPQPQPQPQPPTPKPKPQPQPQPQPQPSTKPKPKPKPPSSSTSETEVESTTESGFISTTTRIITTSIPGKVRPTHTEPSTKPSTEPSTRPKPKPRPRPKPKPSTTTKPTTKPTTKPEDTGTIKGSSMRLLGIDRIELLFMTIAIIFGIWMI